(19) United States
(12) Patent Application Publication
(10) Pub. No.: US 2025/0177710 A1
(43) Pub. Date: Jun. 5, 2025

(54) MEDICAL SYSTEMS FOR TREATING CONDITIONS AND DISEASES OF THE PATIENT'S BODY CAVITIES

(71) Applicant: U.T.T. URINARY TRACT TREATMENTS LTD, Tel Aviv (IL)

(72) Inventors: Eli BEN HAROOSH, Yavne (IL); Asher HOLZER, Raanana (IL); Yosh DOLLBERG, Raanana (IL)

(21) Appl. No.: 19/043,980

(22) Filed: Feb. 3, 2025

Related U.S. Application Data
(63) Continuation-in-part of application No. PCT/IL2024/050659, filed on Jul. 7, 2024.
(60) Provisional application No. 63/526,032, filed on Jul. 11, 2023.
(60) Provisional application No. 63/637,982, filed on Apr. 24, 2024.

(51) Int. Cl. A61M 31/00; A61F 2/00; C07K 16/28

(52) U.S. Cl. CPC A61M 31/002; A61F 2/0018; C07K 16/2818; C07K 16/2896; A61F 2210/0004; A61F 2210/009; A61F 2250/0067; A61M 2202/0007; A61M 2205/0205; A61M 2205/0216; A61M 2210/1082; A61M 2210/1085

(57) ABSTRACT

The present invention provides methods and systems for treating conditions and diseases of a patient's body cavities, including urinary incontinence, transitional cell carcinoma, and other conditions. In particular, the present invention describes a drug delivery system for controlled delivery of drugs to body cavities of a patient, and the use of this system for treatment of the body cavities of the patient, for example, intravesical treatment. Many medications prescribed to patients with urinary tract diseases act locally. In many cases, such medications are administered orally or intravenously and spread through the digestive tract and bloodstream throughout the body, thereby affecting other body systems such as the immune system, kidneys, or liver and causing problematic adverse reactions. For local treatment, the present invention applies the drug directly to the cavity in a minimally invasive procedure.

### CROSS REFERENCE TO RELATED APPLICATIONS

This application is a Continuation-in-part of PCT Patent Application No. PCT/IL2024/050659 having International filing date of Jul. 7, 2024, which claims the benefit of priority of U.S. Provisional Patent Application No. 63/526,032, filed Jul. 11, 2023 and U.S. Provisional Patent Application No. 63/637,982, filed Apr. 24, 2024, the contents of which are all incorporated herein by reference in their entirety.

### TECHNICAL FIELD

In general, the present application relates to the field of medical devices and methods for treating conditions and diseases of patient's body cavities. In particular, the present invention describes a drug delivery system for controlled delivery of drugs to body cavities of a patient, and the use of this system for treatment of the body cavities of the patient, for example, intravesical treatment. In addition, the present invention relates to systems and methods for the treatment of urinary incontinence. The systems of the present invention are characterised by comprising a deformable, biocompatible and biodegradable article designed to be inserted into the patients body cavities.

### BACKGROUND

Many medications prescribed to patients with urinary tract diseases act locally, for example by inhibiting certain receptors that trigger the sudden urge to urinate or stopping the life cycle of cancer cells. In many cases, such medications are administered orally or intravenously and spread through the digestive tract and bloodstream throughout the body, thereby affecting other body systems such as the immune system, kidneys, or liver and causing problematic adverse reactions. Adverse reactions may even cause patients to abandon therapy and instead accept the unpleasant consequences of the disease as the side effects overwhelm the patient.

For local treatment, the drug is applied to the cavity tissue using an injection device. Injecting drugs directly into the body cavity significantly reduces the side effects of drugs. However, direct intracavitary or intraluminal application of a liquid medication may result in short-term tissue effects as the medication is washed out by body fluids. For example, an anti-cancer drug introduced into the bladder through a urethral catheter will likely be eliminated within about an hour, limiting the effect to a negligible level. Therefore, it may be necessary to repeat the procedure several times and periodically reintroduce the urethral catheter to administer the drug, dissolved, for example, in a sodium chloride solution. Because of the need for repeat catheterization, many patients do not choose this type of treatment, although the side effects may be minor. Due to unsatisfactory therapeutic options, there has been a long-felt need for a new method and a new system for drug delivery to a patient's body cavities, for example for the intravesical drug delivery to the bladder.

Another example of a urinary tract disease contemplated by the present invention is urinary incontinence (UI), the involuntary leakage of urine, which is a widespread health issue affecting millions globally, primarily adults and older populations. It impacts quality of life, causing physical discomfort, social embarrassment, and psychological distress. Urinary incontinence can stem from various factors, including weakened pelvic floor muscles (common after childbirth or surgery), neurological conditions (e.g., stroke, spinal cord injury), overactive bladder, enlarged prostate in men, medications, and even chronic coughing. The type of UI determines the most suitable management approach. The common UI types include: stress incontinence (leakage triggered by physical exertion like coughing, laughing, or sneezing); urge incontinence (sudden, strong urge to urinate followed by involuntary leakage); mixed incontinence (a combination of both stress and urge incontinence); and overflow incontinence (difficulty emptying the bladder completely, leading to leakage).

Besides the obvious problem of involuntary leakage of urine that may lead to social isolation and embarrassment, UI can also cause skin irritation and UTIs. Several medical devices, systems and methods exist to manage UI, each with its advantages and limitations. For instance, absorbent products, such as peds and liners, are disposable and have various absorbency levels, but are bulky and inconvenient; pull-up pants, which are more discreet and comfortable than peds, but have limited capacity and are more expensive. Alternative solutions are based on urine collection devices, such as external catheters (for males), but they cause irritation and require careful hygiene; and vaginal pessaries, which are inserted devices designed to support the urethra and bladder neck to reduce stress incontinence, but require proper fitting and may cause discomfort. All the above solutions (and others) offer some level of management, but each has its own limitations. Accordingly, a long-felt need exists for innovative devices, systems and methods to improve the quality of life for people living with urinary incontinence, devices that are comfortable, suitable for all types of UI, discreet and user-friendly, cost-effective and long-lasting.

There are numerous bulk cancers located in the inner body cavities. Ovarian cancer, bladder cancer and rectal cancers are the most common. Some of those cancers are Endometrial cancer type of cancers. Recently some advanced treatments based on (PD-1)-blocking monoclonal antibody has been approved by the FDA for endometrial cancer. Dostarlimab is a monoclonal antibody used as an anti-cancer medication for the treatment of endometrial cancer. Dostarlimab is a programmed death receptor-1 (PD-1)-blocking monoclonal antibody. This drug as well as other similar drugs, is administered through intravenous solution (gxly 500 mg/10 mL). Since this drug is targeting endometrial cancer cells it is used for ovarian, bladder, and GI tract cancers.

Another similar drug that had been most recently approved is pembrolizumab, which is a humanized antibody, more specifically a PD-1 lnhibitor, used in cancer immunotherapy that treats melanoma, lung cancer, head and neck cancer, Hodgkin lymphoma, stomach cancer, cervical cancer, and certain types of breast cancer. It is administered by slow intravenous injection. The only way these types of drugs −(PD-1) blocking monoclonal antibodies—are used is through IV administrations.

Cetrelimab is another drug that can be administered locally into the body cavity. It is a fully humanized immunoglobulin G4 kappa monoclonal antibody that targets programmed cell death protein-1 (PD-1). In human cancer models, Cetrelimab was generated by phage panning against human and cynomolgus monkey (cyno) PD-1 extracellular domains (ECDs) and affinity maturation. It binds to primate and rodent PD-1 ECDs, transfected and endogenous cell-surface PD-1, to inhibit ligand binding.

Despite the inclusion of multiple agents within the prostate cancer treatment landscape, new treatment options are needed to address the unmet needs of patients with local prostate cancer (mCRPC). Although prostate-specific membrane antigen is the only cell-surface target to yield clinical benefit in men with advanced prostate cancer, additional targets may further advance targeted immune, cytotoxic, radiopharmaceutical, and other tumour-directed therapies for these patients. Human kallikrein 2 (hK2) is a novel prostate-specific target with little to no expression in non-prostate tissues. Targeted delivery of drugs for treating cancer has been recognized for quite some time as one of the best modalities of cancer treatment. Intracavitary or intraluminal treatment of drugs has its own advantages.

This disclosure teaches a new way to have both intracavitary or intraluminal delivery and local delivery of targeted drugs for the treatment of prostate cancer. Human kallikrein-2 (hK2) is overexpressed in most prostate cancers, with minimal regular tissue expression or serum secretion. The humanized monoclonal antibody h11B6 exclusively targets hK2 and is suggested to be administered directly into the prostate.

A phase 0 study (NCT04116164) is a first-in-human trial of [111ln]-DOTA-h11B6 to determine the hK2-targeting potential of h11B6, which has shown some potential for treating prostate cancer. At present, this regimen is given through IV administration, with the idea behind it to treat both cells within the prostate and metastasis. The present disclosure teaches the treatment of localised prostate cancer by administrating this drug directly into the tumour proximity within the prostate. This new method aims to achieve an optimum bio distribution of [111ln]-DOTA-h11B6 and determine a suitable monoclonal antibody (mAb) mass that targets mCRPC with minimal non-tumour targeting and without exposing the whole body to this drug.

Anti-CTLA-4 monoclonal antibodies are a class of immunotherapy drugs that work by targeting and blocking CTLA-4 (Cytotoxic T-Lymphocyte Antigen 4), a protein expressed on the surface of T cells. CTLA-4 plays a crucial role in regulating the immune response by acting as a “brake” that dampens T cell activation.

### SUMMARY

In one aspect, the present invention describes a method for treatment of body cavities conditions and diseases of a patient in need thereof, said method comprises inserting a deformable, biocompatible and biodegradable article into a body cavity of a patient with an insertion aid incorporating said article.

In some embodiments, the method for treating a condition or disease of a body cavity of a patient in need thereof comprises an intracavitary or intraluminal insertion into the body cavity a drug delivery system configured to release at least one drug into the body cavity; said drug delivery system comprises:
(1) An insertion aid suitable for insertion of a biocompatible and biodegradable article into the body cavity, and
(2) The biocompatible and biodegradable article within said insertion aid;
wherein said article is either preloaded with at least one drug before placement inside the insertion aid, or placed inside the insertion aid without the drug and then filled with the drug after insertion into the body cavity; said article is deformable and selected from the group consisting of:
   (a) A deformable article designed to be compressed to a size suitable for placing the article into the insertion aid and through the insertion aid into the body cavity of the patient, wherein the article expands to an expanded retention shape upon release from the insertion aid due to its inherent elasticity; and
   (b) A non-deformable article designed to be compressed to a size suitable for placing the article into the insertion aid and through the insertion aid into the body cavity of the patient, wherein the article is preloaded with the drug prior to insertion and expands to an expanded retention shape upon contact with a liquid medium inside the body cavity; and
   wherein the expanded retention shape is suited to retain the article within the body cavity of the patient for an extended release of the drug; said extended release of the drug is governed by a degradation rate of the article, dissolution rate or a diffusion rate of the drug into the liquid medium inside the body cavity, or by pH, pressure, or temperature changes.

In a certain embodiment, said body cavity is bladder and said conditions and diseases are selected from urinary incontinence, overactive and underactive bladder, interstitial cystitis, urinary tract infections, nocturia, bladder cancer and neurogenic bladder; or said body cavity is renal pelvis of a kidney and said disease is transitional cell carcinoma.

In other embodiments, a method for treating or preventing urinary incontinence in a patient comprises:
a) providing a deformable, biocompatible, and biodegradable article comprising a ferromagnetic element or a magnet, wherein the article has a molecular weight of about, 1, such that it is buoyant in urine;
b) placing the article inside the patient's bladder with an insertion aid; and
c) placing an external part comprising a magnet or a ferromagnetic element near the external urethral orifice, wherein:
   when the external part is adjacent to the external urethral orifice, a magnetic force from the magnet or ferromagnetic element of the external part attracts the article to the external urethral orifice to close the orifice and prevent urine leakage; and
   when the external part is moved away from the external urethral orifice (e.g., when removing underwear), the magnetic force is reduced, allowing the article to move away from the urethral orifice and enabling urine to flow.

In this method, the condition or disease is urinary incontinence in a patient; the biocompatible and biodegradable article further comprises a magnetic or ferromagnetic element, or a magnet, and has a molecular weight of about 1, such that it is buoyant in urine; and the method further comprises placing an absorbent pad with an embedded magnet, or magnetic or ferromagnetic element, near the urethral orifice, wherein:
when the article is within the bladder and the absorbent pad is positioned near the urethra, a magnetic force between the pad and the article attracts the article towards the urethral orifice to seal the urethra and prevent urine leakage; and
when the article is within the bladder and the absorbent pad is moved away from the urethral orifice, the magnetic force is reduced, allowing the article to move away from the urethral orifice and enabling urine to flow.

In a further embodiment, said deformable, biocompatible and biodegradable article used in the method for treating or preventing urinary incontinence in a patient is either preloaded with at least one drug before placement inside the insertion aid, or placed inside the insertion aid without the drug and then filled with the drug after insertion into the body cavity.

In another embodiment, said biocompatible and biodegradable article comprises an enteric coating for the extended release of the drug, wherein the extended release of the drug is controlled by the ability of said article to facilitate zero-order diffusion at a rate that is entirely controlled by openings in said coating. In still another embodiment, the rate of the extended release of the drug from the biocompatible and biodegradable article is zero order over at least 24 hours. In yet another embodiment, the extended release of the drug is controlled by the article's ability to facilitate a controlled release amount of the drug over time. This controlled release is achieved through the permeability of the coating, the presence of holes in the coating, and the coating's biodegradability rate.

In yet further embodiment, said biocompatible and biodegradable article is coated with a biocompatible and biodegradable material, and said at least one drug is released from said article through diffusion into the body cavity of the patient at a constant rate over an extended-release duration of at least one day and up to 30 days, and wherein said article starts degrading after the end of the extended-release duration.

The choice of materials for the coating layer of the article of the invention and the degree of the cross-linking of the coating layer enable degradation start from one week after it is inserted into the body cavity and up to three months post insertion. The long release duration enables the utilization of minimal drug dosage to reduce adverse effects, the essentially constant release rate provides an essentially constant drug concentration throughout the drug release duration for the highest efficacy, and the article biodegradability enable natural voiding and improves patient compliance. The combination of the above features provides a multi-faceted solution for extended drug delivery from the article of the present invention.

In yet further embodiment, the article is coated by a thin layer of a biocompatible and non-biodegradable coating material, for example parylene. The layer thickness of such coating is approximately between 5 μm and up to 50 μm and its weight is approximately between 1 mg and 5 mg. When the article material is biodegraded and voided, the thin coating layer loses its mechanical support and is voided naturally.

In another embodiment, the coating of the article is made of the same material as the article core (matrix), but said coating having a different permeability to a drug and different biodegradation rate. As a non-limiting example, the article made from collagen is coated with a different layer of collagen or gelatine having a different permeability to a drug and different biodegradation rate. This can be achieved through varying degrees of cross-linking of the material.

Non-limiting examples of the biocompatible and biodegradable polymer coatings used in the present invention are poly(lactic acid) (PLA), poly(lactic-co-glycolic acid) (PLGA), polycaprolactone (PCL), poly(ε-caprolactone)-based copolymers, chitosan, gelatine, polyhydroxyalkanoates (PHA), polyethylene glycol (PEG), hyaluronic acid, polyglactin, collagen, alginate, and carrageenan. Non-limiting examples of biocompatible and non-biodegradable polymer coatings suitable for sloughing are polytetrafluoroethylene (PTFE), silicone, polyurethane, polycarbonate, and polysiloxane. These materials are biocormpatible and, when applied as a thin coating, can slough off (shed) over time. This sloughing action can be beneficial for controlled drug release or other desired functionalities.

A non-limiting example of the biocompatible and biodegradable article in the present invention is an absorbing, compressible sponge, which is suitable for:
1) absorbing a drug;
2) being compressed and placed in the insertion aid,
3) being released from the insertion aid into the body cavity upon insertion the insertion aid into the body cavity,
4) being expanded inside the body cavity upon contact with a liquid media, or through its inherent elasticity and
5) releasing the drug into the liquid media of the body cavity.

In still another embodiment, the method of the present invention further comprises a step of compressing the sponge and pushing the sponge, while it is compressed, into the insertion aid prior to inserting the insertion aid into the body cavity.

In some embodiment, said drug preloaded into the biocompatible and biodegradable article is in a liquid solution, or mixed with a gel for controlling viscosity, or in a dry soluble form. Non-limiting examples of said biocompatible and biodegradable article are:
(1) a sponge made of collagen;
(2) a sponge made of gelatine;
(3) a sponge made of a combination of gelatine and collagen;
(4) a collagen-containing sponge comprising an absorbable gelatine sponge, collagen, and an active ingredient;
(5) a collagen-containing sponge comprising an absorbable sponge, a reverse gelation biodegradable gel, and the drug;
(6) a foamed absorbable polymeric matrix comprising non-absorbable, drug-carrying microspheres embedded in said matrix is suited for degradation inside the body cavity and release of the microspheres, which are eliminated through the body cavity;
(7) a microsponge or plurality of microsponges comprising porous, microscopic, polymer-based microspheres that are suited for suspending or entrapping the drug and releasing the drug in a sustained flow out of the microspheres;
(8) a plurality of microsphere hydrogel sponges; and
(9) a marine sponge-derived natural sponge.

The aforementioned foamed absorbable polymeric matrix may be composed of poly(D,L-lactide-coglycolide)-copolyethylene glycol di-block copolymer, said copolymer is used to impart a short degradation time to the article. The aforementioned non-absorbable, drug-carrying microspheres may be incorporated into a formulated product selected from a gel, cream, liquid or powder. The aforementioned microsphere hydrogel sponges may comprise poly(trimethylol-propane ethoxylate triacrylate) microspheres cross-linked by a hydrogel, which is formed by a starch-based bifunctional emulsion stabiliser.

In a certain embodiment, said biocompatible and biodegradable article comprises a plurality of biodegradable nanosponges, said nanosponges are nanosized drug carriers with a three-dimensional structure created by crosslinking polymers, and they are suited for providing a controlled drug release pattern with targeted drug delivery. In a particular embodiment, said biocompatible and biodegradable article is comprised of chemical compounds selected from β-cyclodextrins, alginates, carboxymethyl cellulose, chitosan, carrageenans, cross-linked cellulose nanofibers, and collagen, or combinations thereof. Said biocompatible and biodegradable article may be preloaded with the drug by a liquid-liquid suspension polymerisation or a quasi-emulsion solvent diffusion technique.

In a further embodiment, the drug is in a liquid solution (including supersaturated solution), a suspension, a powder, or a form of solid particles.

In a further embodiment, said drug is selected from the group consisting of antineoplastic drugs, anticancer drugs, chemotherapeutic agents, anti-infective agents (such as antimicrobial drugs, antiparasitic agents, antivirals), genitourinary system drugs, anti-inflammatory drugs, analgesics, musculoskeletal system acting drugs, drugs acting on the blood and blood forming organs (such as antihemorrhagics, antithrombotic agents, antianemia drugs), dermatologic drugs (such as antifungals, antiseptic), gastrointestinal system (such as anti-obesity, acid-related disorders), metabolism drugs, neurological drugs, respiratory drugs including nasal drugs, cardio-vascular drugs, ontological drugs, corticosteroids drugs, analgesics drugs, antiparasitic drugs, anaesthetic drugs, botulinum toxin and antibiotics.

In a specific embodiment, said drug is selected from the group consisting of amoxicillin, ceftriaxone, cephalexin, ciprofloxacin, fosfomycin, levofloxacin, amikacin, piperacillin, nitrofurantoin, trimethoprim, sulfamethoxazole, gentamicin, imipenem, meropenem, progesterone, ceftolozane, cefiderocol, plazomicin, neomycin, kanamycin, paromomycin, bacitracin, vancomycin, colistin, polymyxin, amphotericin, lidocaine, paclitaxel, mitomycin C, gemcitabine, gemcitabine mitomycin, quinolone, fluoroquinolone, nadofaragene firadenovec, enfortumab vedotin, sacituzumab govitecan and brilacidin.

In an additional aspect of the present invention, a system for treatment of body cavities conditions and diseases of a patient in need thereof comprises a deformable, biocompatible and biodegradable article designed to be inserted into a body cavity of a patient with an insertion aid incorporating said article. According to this aspect, there are two particular embodiments of the system of the present invention:
I. A system comprising the deformable, biocompatible and biodegradable article without a magnet, or magnetic or ferromagnetic particles, and used for an intracavitary or intraluminal application and sustained release of a drug into the body cavity of a patient; and
II. A system comprising the deformable, biocompatible and biodegradable article with a magnet, or magnetic or ferromagnetic particles, and used for treating or preventing urinary incontinence of a patient.

Thus, the application of the system of the present invention actually depends on the presence or absence of a magnet, magnetic or ferromagnetic particles inside the biocompatible and biodegradable article of the invention. In one embodiment of the present invention, the first system is for intracavitary or intraluminal application and sustained release of a drug into a body cavity of a patient, and comprises:
(1) An insertion aid suitable for insertion of a biocompatible and biodegradable article into the body cavity; and
(2) The biocompatible and biodegradable article;
   wherein the article is either preloaded with at least one drug before placement inside the insertion aid, or placed inside the insertion aid without the drug and then filled with the drug after insertion into the body cavity; said article is deformable and selected from the group consisting of:
(a) A deformable article that can be compressed to a size suitable for placing the article into the insertion aid and through the insertion aid into the body cavity of the patient, wherein the article expands to an expanded retention shape upon release from the insertion aid due to its inherent elasticity; and
(b) A non-deformable article that can be compressed to a size suitable for placing the article into the insertion aid and through the insertion aid into the body cavity of the patient, wherein the article is preloaded with the drug prior to insertion and expands to an expanded retention shape upon contact with a liquid medium inside the body cavity; and
   wherein the expanded retention shape is suited to retain the article within the body cavity of the patient for an extended release of the drug; said extended release of the drug is governed by a degradation rate of the article, dissolution rate or a diffusion rate of the drug into the liquid medium inside the body cavity, or by pH, pressure, or temperature changes.

In another embodiment, the second system is for treating or preventing urinary incontinence in a patient and comprises:
(i) An absorbent pad with an embedded magnet or ferromagnetic element, positioned near the urethral orifice; and
(ii) The biocompatible and biodegradable article for insertion into the patient's bladder, the article comprising a ferromagnetic element or a magnet and having a molecular weight of about 1, such that it is buoyant in urine;
   wherein the system is configured such that,
when the absorbent pad is positioned near the urethra, a magnetic force between the external part and the article attracts the article towards the urethral orifice to seal the urethra and prevent involuntary urine leakage; and
when the absorbent pad is moved away from the urethral orifice, the magnetic force is reduced, allowing the article to move away from the urethral orifice and enabling urine to flow.

In a particular embodiment of the present invention, the insertion aid is a catheter comprising a part of an endoscope or cystoscope with a working cannel where the biocompatible and biodegradable article is compressed inside the working channel.

In a further embodiment, the biocompatible and biodegradable article of the second system designed for treating or preventing urinary incontinence is water-sealed and free-floating in a urinary bladder. This article comprises a core material having a specific gravity that is less than a specific gravity of water, said material allowing for the internal compartment to be buoyant in aqueous fluids. Non-limiting examples of said core material are foam, porous solid, liquid or gaseous material.

In an additional embodiment, the biocompatible and biodegradable article of the second system designed for treating or preventing urinary incontinence further comprises a flexible funnel holding said biocompatible and biodegradable article, said funnel comprises a distal end designed to be positioned in the patient's urethra, and a proximal end designed to reside within the urinary bladder at the urethral orifice, wherein said funnel comprises a mesh at its broad part designed to prevent the biocompatible and biodegradable article from distancing from the urethral orifice and maintain it close to the urethral orifice to enable the creation of a magnetic pull between said ferromagnetic element and said magnet.

In one embodiment, said flexible funnel of the biocompatible and biodegradable article of the second system designed for treating or preventing urinary incontinence has an expandable tip, such as a disk or a malecot-flower, designed to expand within the urethra to therefore anchor said funnel in place. In yet further embodiment, said external part is designed to be inserted in or be a part of the patient's underwear.

In a further aspect of the present invention, a method for the preparation of the biocompatible and biodegradable article of claim the invention is selected from liquid-liquid suspension polymerisation, quasi-emulsion solvent diffusion, multiple-emulsion solvent diffusion, porogen addition method, lyophilisation and ultrasound technique.

In yet further embodiment of the invention, a kit for use in a intracavitary or intraluminal application and sustained release of a drug into the body cavity of a patient comprises the first (drug delivery) system of the invention, and instructions for use of said drug delivery system. In still another embodiment, a kit for use in treating or preventing urinary incontinence of a patient, comprising the second system of the invention, and instructions for use of said system.

In addition, the kits of the present invention comprise instructions in a form of an instruction manual, which is generally written instructions, although an electronic storage medium, such as an optical disc, a flash drive or a link to a cloud containing the instructions, is also acceptable.

In a specific embodiment, the present invention provides the aforementioned method, system and kit for treatment of body cavities conditions and diseases of a patient in need thereof, where said drug introduced with the deformable, biocompatible and biodegradable article of the invention into a body cavity of a patient is amikacin.

In a specific embodiment, the present invention provides the aforementioned method, system and kit for treatment of body cavities conditions and diseases of a patient in need thereof, where said drug introduced with the deformable, biocompatible and biodegradable article of the invention into a body cavity of a patient is gemcitabine.

In some embodiments, the drug used in the drug delivery system of the present invention is a PD-1 inhibitor. The drug delivery system of the present invention is thus suitable for local administration of a PD-1 inhibitor for cancer treatment, thereby reducing side effects relating to intravenous administration and improve efficacy. Some non-limiting examples of PD-1 inhibitors include dostarlimab and pembrolizumab.

The invention also contemplates embodiments where the the biocompatible and biodegradable article, loaded with a drug, is implanted directly into a body tissue, rather than a body cavity. In particular, the system may be implanted into the male prostate.

The drug delivery system of the present invention is thus suitable for local administration of a humanized monoclonal antibody (mAb) h11B6 that exclusively targets human kallikrein 2 (hK2) trial for the treatment of prostate cancer, thereby reducing side effects relating to intravenous administration and improve efficacy. It can be conjugated with a radioactive material, such as iodine-131 or iron-55, or any suitable anti-cancer drug.

In other embodiments, the invention provides for the direct injection of the h11B6 antibody (mAb) into the prostate tissue. This is achieved by utilizing the sponge delivery system of the present invention as a carrier for the antibody.

In yet another embodiment, the drug used in the drug delivery system of the invention is an anti-CTLA-4 monoclonal antibody. The drug delivery system of the invention is thus suitable for local administration of anti-CTLA-4 monoclonal antibodies for the treatment of bladder cancer, thereby reducing side effects relating to intravenous administration and improve efficacy. Non-limiting examples of anti-CTLA-4 monoclonal antibodies include ipilimumab and tremelimumab.

In some embodiment, anti-CTLA-4 monoclonal antibodies are administered in combination with an anti-PD-1 antibody. This can help to further enhance the immune response against cancer cells. In yet another embodiment, the anti-PD-1 antibody is nivolumab. In a further embodiment, the anti-CTLA-4 monoclonal antibody is administered intravesically. In certain embodiments, anti-CTLA-4 monoclonal antibodies are administered in conjunction with a chemotherapeutic agent. This may involve concurrent administration or a combination regimen where the antibody and chemotherapeutic drug are administered sequentially.

### BRIEF DESCRIPTION OF THE DRAWINGS

Disclosed embodiments will be understood and appreciated more fully from the following detailed description taken in conjunction with the appended figures. The drawings included and described herein are schematic and are not limiting the scope of the disclosure. It is also noted that in the drawings, the size of some elements may be exaggerated and, therefore, not drawn to scale for illustrative purposes. The dimensions and the relative dimensions do not necessarily correspond to actual reductions to practice of the disclosure.

FIG. 1A illustrates the article of the invention in the form of the expanded sponge with the insertion aid.

FIG. 1B illustrates the deformable, biocompatible and biodegradable article of the invention in its expanded mode in a form of an open-cell sponge.

FIG. 1C illustrates the deformable, biocompatible and biodegradable article of the invention in its expanded mode in a form of a closed-cell sponge with a drug located inside the closed cells of the sponge.

FIG. 1D schematically shows the compressed article inserted in the urethral catheter.

FIG. 1E schematically shows the urethral catheter with the expanded article being pushed out into the bladder.

FIG. 1F schematically shows the article released into the bladder.

FIG. 2 schematically shows an endoscope with a catheter inserted into the bladder, where the compressed article is located in its working channel.

FIG. 3 schematically shows a catheter inserted into the bladder. The compressed article can be inserted into it and pushed into the bladder.

FIGS. 4A and 4B schematically show a bladder holding the system of the present invention for the treatment of urinary incontinence.

FIG. 5A schematically shows the article (100) of the present invention comprising numerous ferromagnetic/paramagnetic beads within it.

FIG. 5B schematically shows the article (100) of the invention shown in FIG. 5A in a compressed/squeezed mode.

FIGS. 6A and 6B schematically show some configurations of the article (100) of the invention.

FIGS. 7A and 7B schematically show the external part of the system of the present invention used for the treatment of urinary incontinence.

FIGS. 8A and 8B schematically show an embodiment of the invention in which the article (100) of the invention is held within a dedicated funnel.

FIGS. 9A and 9B schematically show two types of funnel structures:

FIG. 9A illustrates a funnel with a disk portion at its narrow side; and

FIG. 9B illustrates a funnel with a malecot-flower at its narrow side.

FIG. 9C is an illustration of how the article (100) of the invention closes the funnel when a magnetic force is applied.

FIG. 10 schematically shows a ureteral catheter, called a “double J” or “pigtail”, which is covered with a thin layer of sponge filled with a mixture of drug and gel.

### DETAILED DESCRIPTION

In the following description, various aspects of the present application will be described. For purposes of explanation, specific details are set forth in order to provide a thorough understanding of the present application. However, it will also be apparent to one skilled in the art that the present application may be practiced without the specific details presented herein. Furthermore, well-known features may be omitted or simplified in order not to obscure the present application.

The term “comprising”, used in the claims, is “open ended” and means the elements recited, or their equivalent in structure or function, plus any other element or elements which are not recited. It should not be interpreted as being restricted to the means listed thereafter; it does not exclude other elements or steps. It needs to be interpreted as specifying the presence of the stated features, integers, steps or components as referred to, but does not preclude the presence or addition of one or more other features, integers, steps or components, or groups thereof. Thus, the scope of the expression “a system or device comprising x and z” should not be limited to systems and devices consisting only of components x and z. Also, the scope of the expression “a method comprising the steps x and z” should not be limited to methods consisting only of these steps.

Unless specifically stated, as used herein, the term “about” is understood as within a range of normal tolerance in the art, for example within two standard deviations of the mean. In one embodiment, the term “about” means within 10% of the reported numerical value of the number with which it is being used, preferably within 5% of the reported numerical value. For example, the term “about” can be immediately understood as within 10%, 9%, 8%, 7%, 6%, 5%, 4%, 3%, 2%, 1%, 0.5%, 0.1%, 0.05%, or 0.01% of the stated value. In other embodiments, the term “about” can mean a higher tolerance of variation depending on for instance the experimental technique used. Said variations of a specified value are understood by the skilled person and are within the context of the present invention. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges, for example from 1-3, from 2-4, and from 3-5, as well as 1, 2, 3, 4, 5, or 6, individually. This same principle applies to ranges reciting only one numerical value as a minimum or a maximum. Unless otherwise clear from context, all numerical values provided herein are modified by the term “about”. Other similar terms, such as “substantially”, “generally”, “up to” and the like are to be construed as modifying a term or value such that it is not an absolute. Such terms will be defined by the circumstances and the terms that they modify as those terms are understood by those of skilled in the art. This includes, at very least, the degree of expected experimental error, technical error and instrumental error for a given experiment, technique or an instrument used to measure a value.

As used herein, the term “and/or” includes any and all combinations of one or more of the associated listed items. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

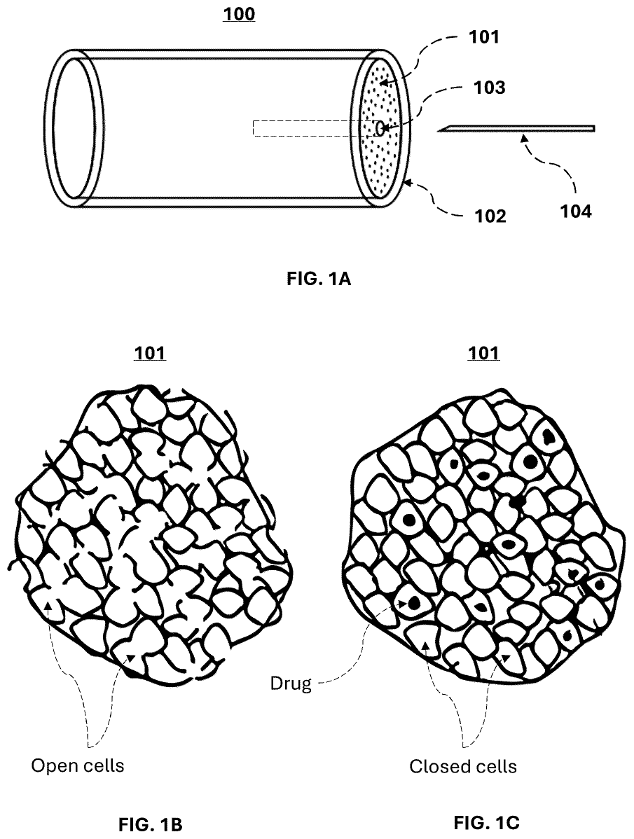

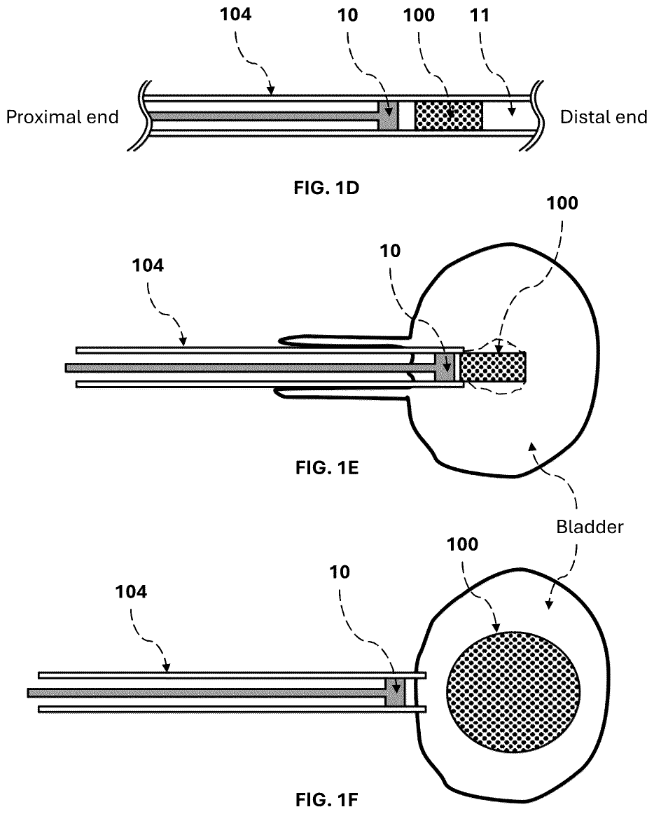

The present invention describes a method for treating conditions and diseases of patients body cavities comprising inserting a deformable, biocompatible and biodegradable article into a body cavity of the patient. A non-limiting example of this article of the invention is schematically shown in FIG. 1A, which illustrates the article in the form of the expanded sponge for carrying a drug. This exemplary article (100) of the present invention comprises core sponge (101), coating (102) and a trough or non-through hole (103), as shown in the figure, made along the middle axis of the core sponge (101) for inserting insertion aid (103), for example a needle (syringe) or tube.

FIG. 1B schematically shows the core sponge (101) of the article in its expanded shape in a form of an open-cell sponge. The core sponge (101) may be prepared from the biocompatible and biodegradable cross-linked sponge material or cross-linked polymeric coating, and incorporating the drug inside its closed cells. FIG. 1C schematically shows core sponge (101) in its expanded shape in a form of a closed-cell sponge with a drug located inside the closed cells of the sponge. All methods and systems of the present invention are characterised by containing this deformable, biocompatible and biodegradable article, which will be described in more detail below.

In one embodiment, a drug delivery system for an intracavitary or intraluminal delivery of a drug into a body cavity of a patient comprises:
(1) An insertion aid suitable for insertion of a biocompatible and biodegradable article into the body cavity, and
(2) The biocompatible and biodegradable article within said insertion aid;
   wherein said article either preloaded with at least one drug before placement inside the insertion aid, or placed inside the insertion aid without the drug and then filled with the drug after insertion into the body cavity; said article is deformable and selected from the group consisting of:
(a) A deformable article capable of being compressed to a size suitable for placing the article into the insertion aid and through the insertion aid into the body cavity of the patient, wherein the article expands to an expanded retention shape upon release from the insertion aid due to its inherent elasticity; and
(b) A non-deformable article capable of being compressed to a size suitable for placing the article into the insertion aid and through the insertion aid into the body cavity of the patient, wherein the article is preloaded with the drug prior to insertion and expands to an expanded retention shape upon contact with a liquid medium inside the body cavity; and
   wherein the expanded retention shape is suitable for retaining the article within the body cavity of the patient for an extended release of the drug; said extended release of the drug is governed by a degradation rate of the article, dissolution rate or a diffusion rate of the drug into the liquid medium inside the body cavity, or by pH, pressure, or temperature changes.

In some embodiments, the method of the present invention is suitable for an intracavitary or intraluminal application and sustained release of a drug into a body cavity of a patient, comprising inserting into the body cavity a deformable, biocompatible, and biodegradable article that is configured to release at least one drug into the body cavity,
wherein the article is either preloaded with at least one drug before placement inside the insertion aid, or placed inside the insertion aid without the drug and then filled with the drug after insertion into the body cavity; said article is deformable and selected from the group consisting of:
(a) A deformable article that can be compressed to a size suitable for placing the article into the insertion aid and through the insertion aid into the body cavity of the patient, wherein the article expands to an expanded retention shape upon release from the insertion aid due to its inherent elasticity; and
(b) A non-deformable article that can be compressed to a size suitable for placing the article into the insertion aid and through the insertion aid into the body cavity of the patient, wherein the article is preloaded with the drug prior to insertion and expands to an expanded retention shape upon contact with a liquid medium inside the body cavity; and
   wherein the expanded retention shape is suited to retain the article within the body cavity of the patient for an extended release of the drug; said extended release of the drug is governed by a degradation rate of the article, dissolution rate or a diffusion rate of the drug into the liquid medium inside the body cavity, or by pH, pressure, or temperature changes.

The release duration of the drug is controlled by the following factors: the mechanism of degradation of the biocompatible and biodegradable article, the mechanism of diffusion of the drug into the liquid media inside the body cavity, dissolution rate or a diffusion rate of the drug into the liquid media inside the body cavity, degradation of a gel that is pre-mixed with the drug, the diffusion of the drug out of the gel, specific coating and shape of the carrier, the effects of pH, pressure or temperature changes on the said degradation and diffusion.

When the main release mechanism is carrier degradation, the drug concentration in the body cavity throughout the treatment duration is controlled by the carrier surface area. When the main release mechanism is diffusion from the carrier that drug concentration is controlled by the diffusion port size. Thus, this method enables a control over the drug release rate from the carrier and into the body cavity.

It is known in the art that optimal pharmacokinetics is correlated with the rate of drug release. Most drugs are delivered as immediate-release formulations that lead to a rapid increase in systemic drug concentration, adverse effects, low bioavailability, or undesirable pharmacokinetics. Drug delivery systems featuring first-order release kinetics provide improved pharmacokinetics but are not ideal for drugs with short biological half-lives or small therapeutic windows. Zero-order drug delivery systems release drug at a constant rate, thereby maintaining drug concentrations within the optimal therapeutic window for an extended period of time and limiting adverse effects. When a biocompatible and biodegradable article of zero-order diffusion mechanism is inserted, the drug dose inside a bladder increases linearly and decreases abruptly every several hours, at urination. For the short duration when the bladder is almost empty, the release of drug from the carrier is slowed. Since the body naturally generates urine at a rate of between 1 and 2 millilitres per minute, the drug dose reverts to the linear increase within 1 to 10 minutes.

Thus, when the biocompatible and biodegradable article containing a drug of zero-order diffusion mechanism is inserted, the drug concentration in the bladder tissue remains essentially constant throughout the drug release period, except for several minutes following urination every several hours. Since the bladder wall is essentially collapsed over the article, the residual and the newly generated urine in the bladder will renew the tissue exposure to the same constant drug concentration within several minutes.

The drug dosage to be released depends on the drug and clinical application. The pharmacokinetics for each drug dictates the minimal therapeutic level and the toxic level. It is known in the art that the effect of a prolonged exposure of the tissue to the drug is similar or better than short exposure to higher drug dosage. When cancer is treated, the cancer cells are susceptible to the chemotherapeutic agent only during part of their cycle time, thus the longer the drug is exposed to the cancer cells, the treatment is more effective. The present invention teaches how to control a zero-order release of the drug or any other elution curve.

The drug delivery system of the present invention enables a prolonged drug treatment at an essentially constant concentration level that is above the minimal therapeutic level for the specific drug while limiting the risk of that level reaching the toxic concentration level. The adaptation of the carrier to a specific release rate can be achieved, in a non-limiting example, by adaptation of the release port area. In a non-limiting example 80 milligrams of mitomycin C can be used for the treatment of bladder cancer by their release at a constant rate for over 80 hours, while yielding a constant concentration of 1×10⁻⁶.

In specific embodiments shown in FIG. 1A, the drug carrier, for example sponge, is sealed by an essentially watertight coating (102) with one or more openings that together create a diffusion port with a controlled and limited area. Thus, the diffusion rate depends on the area of the diffusion port leading to an essentially constant diffusion rate. In specific embodiments, the coating (102) is a membrane with specific permeability that enables the passage of water but limits the passage of certain drugs.

In specific embodiments, the carrier is a biocompatible and biodegradable sponge suited for:
1) absorbing liquids such as drugs;
2) being compressed and placed inside an insertion aid such as a catheter,
3) being released from the insertion aid into a body cavity such as a bladder upon,
4) being expanded inside the body cavity upon insertion into the body cavity, and
5) releasing the drug into the liquid media of the body cavity.
6) degrade and be expelled from the body cavity after a period of time that is longer than the drug release, for example after 1 month.

Non-limiting examples of such sponges are sponges made of collagen or gelatine. Sponges like that are commercially available and mostly used in applications such as haemostasis elements, an absorbable hemostat made by oxidized regenerated cellulose (ORC), and a regenerative collagen dental sponge for surgical procedures.

In certain embodiments, the sponge drug carrier is coated with less permeable material, for example by a highly cross-linked layer of the sponge material, such as collagen or gelatine or by a thin layer of oil-based or silicone-based ointment, such as a vaseline, or by chemical vapor deposition of very thin polymer such as parylene, having a thickness of about 5 to 20 microns, or by mechanically coating with other polymers, such as polyvinyl alcohol (PVA) or paraffin; such coating layer minimizes the diffusion of drug from the sponge while mostly retaining the ability of the sponge to be compressed, thereby allowing to control the diffusion of the drug out of the carrier.

In some embodiments utilizing a coated sponge carrier, the elution of drug from the coated sponge occurs essentially by pure diffusion. The diffusion rate is essentially dictated by the number and area of the openings in the coating, such that the diffusion rate is essentially constant, known as zero-order diffusion. A constant rate of drug elution minimizes the risk of adverse events due to drug concentration that is higher than optimal as may occur in extended drug elution systems that rely on carrier degradation or higher-order diffusion.

Thus, the coating layer controls diffusion if it has different drug permeability. The main idea of coating is to provide zero-order release. If, for example, the coating seals the drug, then the release of the drug should only occur through an opening made in it, providing a zero-order release. After some time, complete degradation followed.

In certain embodiments, the insertion aid (104) is the urethral catheter that is either designed or adapted to store a compressed sponge carrier, push it out into the bladder and enable the injection of a liquid such as drug into the sponge. For example, this can be achieved by the use of a rigid stylet that is adapted to push the sponge, with a thin tube that is fitted through the stylet stem and into substantially most of the length of the sponge carrier. Reference is now made to FIG. 1D describing the urethral catheter (104) as the insertion aid fitted to be inserted into a bladder, where a compressed sponge (100) is located in its distal end. A pushing rod Stylet (10) is inserted into the catheter channel (11).

In some embodiments, the sponge (100) is shaped with a narrowing proximal end to facilitate the insertion of the catheter (104) over the male prostate. In other embodiments, the proximal end of the catheter (104) is shaped for that insertion and with an ability to push that proximal end out of the sponge insertion route.

Reference is now made to FIG. 1E schematically showing the catheter (104) with the sponge article (100) being pushed out into a bladder. The catheter (104) is inserted into an empty bladder and then the compressed sponge (100) is pushed by rod Stylet (10) into the bladder. In some embodiments of the invention, a drug is further embedded in the sponge (100) prior to insertion of the insertion aid (104) into the body cavity (bladder). In other embodiments, the insertion aid (104) with the sponge (100) inside but without the drug in the sponge (100) is initially inserted into the body cavity, and it is filled with the drug after it is inserted into the body cavity. The compressed sponge (100) may be compressed into an insertion aid (104) and then inserted into an empty bladder in a compressed mode, and the drug solution is injected into the sponge (100), or into the empty bladder, thereby making the sponge (100) to absorb the drug solution while in the bladder, and it is expanded due to the absorption of the solution.

Thus, in another embodiment, the compressed sponge (100) is inserted into the catheter (104) and pushed mostly out of the catheter into an emptied bladder. Then, as illustrated in FIG. 1E, the drug solution is injected into the expanding sponge (100), while this sponge is partially still inside the catheter (104). At this point, the sponge (100) is pushed all the way out of the catheter (104). In a specific embodiment, the aforementioned parylene coating of the sponge (100) significantly reduces the friction between the sponge (100) and the catheter (104) to enable the sponge manipulation.

To sum up, FIG. 1E shows that the sponge (100) is already at the distal part of the catheter (104), in a semi-expanded mode. At this stage, there are two alternatives that can be considered:
1. The first alternative is that the sponge (100) is pushed all the way into an empty bladder by pushing rod Stylet (10). An insertion aid (104), for example a syringe or tube, is then inserted through a hole (103), which is seen in FIG. 1A, but not shown here, inside the pushing rod (10). Alternatively, the rod (10) is taken out, and a syringe with the drug is attached into the proximal side of the catheter (not described in the figure). After that, a drug is injected into the empty bladder and being absorbed in the expanded sponge.
2. In the second alternative, the process is similar, but the drug is inserted into the semi expanded sponge (100) seen in FIG. 1E. The drug is then injected into the sponge (100). This way the process is less dependent on the emptiness of the bladder in case some urine still exists there. In yet further embodiment, the pushing rod (10) is a needle with an expanded pedestal in its distal side, so the drug can be injected through the pushing rod (10).

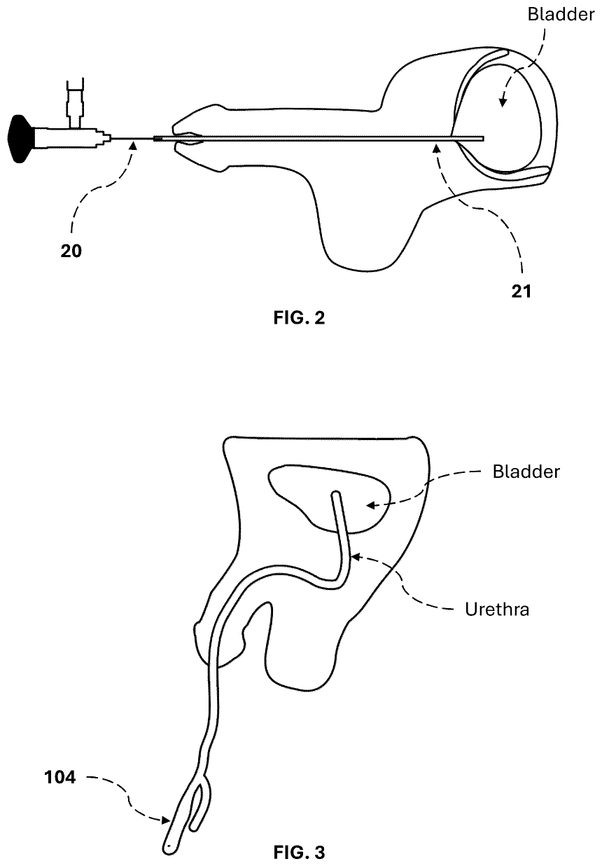

FIG. 1F schematically shows the expanded article (100) of the invention released into the bladder. The expanded article (100) carries a drug within it for extended release. In some embodiments, the drug is pre-mixed with a gel as described below, but not shown in this figure. Reference is now made to FIG. 2 schematically shows an endoscope or a cystoscope (20) inserted into a bladder. A compressed sponge is inserted into working channel (21). After the placement of cystoscope (20) into the empty bladder, the sponge is pushed into the bladder by pushing a rod similar to the rod (10) of FIG., ID, not shown in the present figure. FIG. 3 illustrates a catheter (104) inserted into the bladder through urethra. While this figure demonstrates a non-limiting example of a male anatomy, it may be a female as well.

According to a further aspect, the present invention describes a drug delivery system for delivery of drugs to the bladder of the patient, said drug delivery system comprises:
An insertion aid suitable for insertion into the bladder, and
a biocompatible and biodegradable article placed inside said insertion aid suitable for the intravesical administration of the drugs; and
a retrieval-device suitable for extracting the biocompatible and biodegradable article from the bladder.

Said biocompatible and biodegradable article is either preloaded with drug prior to being placed in the insertion device or is filled with the drug after being completely inserted into the bladder by the insertion device or partially inserted into the bladder by the insertion device. The pushing rod (10) can be made around an injecting needle enabling injecting the drug or and the drug mixed with gel into the sponge. The biocompatible and biodegradable article includes magnetic of ferromagnetic particles. Said retrieval device comprises a magnet and a collapsible grip mechanism. The grip mechanism is collapsed when the retrieval device is inserted into the bladder. Post insertion, the grip mechanism is opened, the biocompatible and biodegradable article is magnetically attracted to the retrieval device, with no need for visual aids and the grip mechanism is closed to attach and compress the biocompatible and biodegradable article and enable its removal from the bladder.

The retrieval device enables the removal of a non-biodegradable biocompatible and biodegradable article for the patient's safety and/or for the reloading of the biocompatible and biodegradable article with drug that can be the same as the original drug or a different drug.

In a non-binding example, the act of locating and attaching the biocompatible and biodegradable article to the said retrieval device can be externally verified either electronically or magnetically.

In specific embodiments, the drug is delivered mixed with a certain gel for controlling its viscosity and the drug release from the sponge. Such gel is described in non-limiting examples in the following patent documents by the present inventors: U.S. Pat. Nos. 9,540,407, 9,801,854, 9,884,028, 9,950,069, 10,039,832, US 2017/0143833, and US 2017/0112935, A non-limiting example is a viscoelastic gel with a reverse rheology property that is already mixed with drug, such that when at low temperature the gel is a low viscosity liquid, while at body temperature it is an elastic semi-solid gel material that can retain the drug mixed into it. In more detail, the mixture of this gel and the drug is kept at low temperatures such as below 8 degrees centigrade, where it is in a low viscosity mode. The cold mixture is then injected into the sponge that is already pushed mostly into the bladder. The gel solidifies inside the sponge at body temperature thus the sponge is filled with drug that is embedded in high viscosity gel that controls the diffusion rate of the drug out of the sponge. In this example, the drug pharmacokinetics

Pharmacokinetics of the drug that is released from the biocompatible and biodegradable article is governed by a degradation rate of the biocompatible and biodegradable article, dissolution rate or a diffusion rate of the drug into the liquid media inside the body cavity, or by pH, pressure, temperature changes, or the degradation rate of a second biocompatible and biodegradable article, such as a gel.

Providing the required pharmacokinetics of drug into the body cavity is very important for obtaining an effective treatment, and while reducing the side effects and the adverse effects that are correlated to the peak drug concentration. A regulated release of constant drug concentration is the optimum for treating the bladder where after every voiding the concentration falls to zero and increased over time until the next urination. In other body cavities usually, a constant release is the optimum way of treatment. This patent teaches regulating the elution rate by activating a zero-order diffusion of drug from the sponge. Zero-order diffusion is achieved by coating the sponge fully with watertight material and leaving controlled openings in the coating so that the diffusion rate is essentially correlated to the openings area. In other embodiment the elution rate is controlled by different features of the sponge carrier that control the biodegradation; among them: sponge material biodegradation speed, size of the sponge cells and consequently the three-dimensional sponge wall area, different coating materials that have different degradation rate and different permeability, having the drug embedded in a gel with known dissolution rate and viscosities. And also controlled by the geometry of the sponge carrier since the diffusion rate depends on the ratio of volume to surface area of the carrier.

In some embodiments, when treating bladder cancer, it is important to expose cancer cells to anti-cancer drugs for as long as possible, since chemotherapy attacks cancer cells preferentially at certain stages of their life cycle, stopping the cells from dividing and causing them to die. According to statistics, the likelihood of such an encounter between a chemotherapy drug molecule and a cancer cell increases with the time the drug remains in the bladder. Thus, long-term use of chemotherapy is necessary to enhance the killing effect and, therefore, for a positive clinical outcome. Since the cycle length of bladder cancer cells ranges from 12 to 36 hours (multiplier), increasing the exposure time from less than an hour to more than 36 hours (and ideally two or three times this duration) increases the effectiveness of killing cancer cells. by several orders of magnitude.

In some embodiment the drug used is gemcitabine or mitomycin or valrubicin which are chemotherapeutic drugs for the treatment of bladder cancer, A total of 75 milligram of drug is loaded into the carrier. The carrier contains a sponge mixed with gel, coated with a thin layer of about 0.4 millimeter all around with one opening where the injected needle was placed in order to load the drug. During the first 5 days the drug release is governed mainly through a diffusion through the opening, since the biodegradation of the cover surface is much longer than 5 days, up to 21 days. Since urination typically takes place every 3 hours, thus for example between 6 at the morning and midnight, there will be 6 urinations and overnight of addition 6 hours. The drug release will be about 15 milligram every 24 hours, over 5 days a total of 60 milligram out of the 75 milligrams that are preloaded in the carrier will be diffused out. The remaining 15 milligrams will be eventually also released by the time of a total degradation takes place, for the remaining of the 21 days. This means that on the average every urination that happens about every 3 hours and contain about 200 cc of urine a total of 2 mg will be released. The concentration will be very stable since the urine accumulates in the bladder with the rate of the diffusion out of the carrier.

Yet in another embodiment, when the goal is to treat urine tract infection, and the drug is certain antibiotic, such as amikacin, a total dosage of 100 milligram is released over 8 days. Administrating the drug directly into the urinary tract, and more even directly into the bladder, enables using drugs that at the present are rarely being used due to the exposure to the system. When treating chronic urinary tract infection, there is for example a very effective antibiotic—amikacin—that is very rarely used since it is very nephrotoxic. The technology described here is enabling using drugs and administrating them locally exposing the target organ for a long period of time to the drug with minimum to none getting into the system. This enables reduction of the total amount of drug used, more effective treatment, reduced side effects and adverse effects, and limiting the whole-body exposure to the drug used.

In some embodiments, the body cavity is the bladder and said conditions and diseases are urinary incontinence, overactive and underactive bladder, interstitial cystitis, urinary tract infections, nocturia, bladder cancer, neurogenic bladder, and others.

In some embodiments, the body cavity is the renal pelvis of a kidney, where urine produced is collected, and said conditions and diseases are cancer and other kidney diseases. In this case, the insertion aid of the invention is a nephrostomy tube, which is a catheter that's inserted through the skin and into the kidney. Through this tube, the sponge carrier is delivered into the kidney, preferably into the calyx proximity.

As seen in FIGS. 1B and 1C, the carrier may be made of an open-cell sponge, a close-cell sponge, or a mix of an open-cell sponge and a close-cell sponge. All these options are either with a magnet embedded within the sponge or without the magnet. In one embodiment, a magnet is inserted within the sponge. In such a case a mix of an open cell sponge and a closed cell filled with air has an advantage since the goal is to have the carrier with a specific weight as close to one, in order to avoid bouncing in the bladder, and to avoid the sponge located in the bladder neck thus disrupting natural voiding. The method of the present invention using the sponge with the magnet will be described below.

In other embodiments, the biocompatible and biodegradable article is an absorbing sponge suited for:
1) absorbing the drug;
2) being compressed and placed in the catheter,
3) being released from the catheter into the body cavity upon insertion the catheter into the body cavity,
4) being expanded inside the body cavity upon release from the catheter, and
5) releasing the drug into the liquid media of the body cavity.

In a particular embodiment, the biocompatible and biodegradable article is a closed cell sponge, or a mix of closed cells and open cells (see FIGS. 1B and 1C) where the drug is preloaded or embedded into it. Such a sponge is inserted into the catheter in a compressed shape suitable for placing the sponge into the catheter. The catheter delivering this compressed sponge is inserted into a body cavity of a patient with the insertion aid. The sponge with a liquid solution delivered through a catheter is then released in an expanded retention shape formed upon being released into the body cavity, or upon a contact of the sponge with a liquid media inside the body cavity, or through its inherent elasticity. It remains expanded inside the body cavity and is retained for a prolonged time within the body cavity of the patient for a prolonged drug release duration. The sponge can be removed from the body cavity by a simple catheterization process, by a specific retrieval device or degrade and be voided naturally or can be degraded over time. The release of the drug from the biocompatible and biodegradable article may be extended over a range of several hours to several weeks.

The drug release profile and duration time depends on the drug used, the clinical indication and the requirement for the drug dosage to be above minimum therapeutic level and the maximum tolerability level. In some embodiments it can be from several hours like 1, 2, 3, and up to 24 hours and several days like 1, 2, 3, and up to 28 days and more.

In one embodiment, a magnet is inserted within the carrier creating the ability to retrieve the sponge at any time during the treatment period by applying a magnetic retrieval device, without the need for a visual or endoscopic assistance. The ability to retrieve the carrier enables simple and rapid termination of the drug treatment for safety or efficacy causes.

In one embodiment, that carrier is a sponge that has both an open cell sponge part and a closed cell sponge part that is filled with air. Such an article is designed to have a specific weight close to one. Such a sponge has the advantage of preventing bouncing in the bladder, while avoiding the sponge being stuck in the bladder neck thus interrupting natural voiding.

Another way to accelerate or to terminate the treatment is to insert into the bladder a solution with a certain enzyme that accelerates the degradation rate of the sponge. The type of enzyme depends on the sponge material. In a non-limiting example, such enzymes include bacterial collagenases that cleave helical regions of fibrillar collagen molecules under physiological conditions. Certain matrix metalloproteinases (MMPs) are known to catalyse degradation of interstitial collagen fibrils.

The method of the present invention allows to control the extended-release time of the drug and the exposure of the inner tissue to the drug, thus increasing the efficacy of the treatment.

The drug release profile is controlled by five mechanisms: a) the diffusion of the drug out of the sponge, b) the biodegradation rate of the sponge, c) the shape of the sponge d) the biodegradation of the gel when mixed with the drug e) the coating of the sponge or part of it with various materials having different biodegradation and permeability chosen to provide desired drug elution rate. The release profile of the drug thus can be as non-limited examples from 30 minutes, 1 hour, 5 hours 1 day, 3 days, 10 days, or longer. The preferred release profile depends on the clinical indication and therapeutic agent. For example, for the treatment of bladder cancer, it is beneficiary to expose the bladder wall to chemotherapy for a duration that is longer than the cell cycle or multiplication time of cancer cells that is 12 to 36 hours. For yet another example, for antibiotics treatment of bladder infection, 8 days are necessary for specific antibiotics while 4 days suffice for others.

In a non-limiting example, the size and geometry of the sponge can be designed to influence the release profile of the drug and/or the bladder functionality. In a non-limiting example, the sponge geometry can be a ball sphere, a cylindrical shape, and a toroidal shape that influences pharmacokinetics and prevents the sponge from being stuck in the bladder neck. Size of the expanded sponge may be from 1 cubic centimetre, up to several cubic centimetres.

In some embodiments, the drug is selected from a group of anticancer drugs. The advantage of the method of the present invention delivering the anti-cancer drugs is that such drugs used in chemotherapy are capable of killing cancer cells mainly during the short time of the cells' replication, thus long-term exposure of the cancer cells to anti-cancer drugs enhances the anti-cancer effect.

If the treated body cavity is bladder, the drugs used in the method of the present invention may be antibacterial drugs for the treatment of urinary tract infections. The proposed administration method delivers the drug into the bladder intravesically and lets it be slowly released directly to the bacteria that are there. While for systemic drug administration the drug is carried by the blood, then has to pass through the blood system and the urothelium to reach the bacterial biofilms that are located on the urothelium. The present invention enables several existing drugs to notably enhancing their clinical effect and to expand their indications.

Additional advantages of intravesical drug administration with extended release, are:
the overall body system is not exposed;
lower drug dosage is required in order to reach a therapeutic level compared to the systemic administration of the same drug; Low peak dosages are associated with lower adverse reactions; therefore, the present invention can potentially reduce adverse effects by reducing the peak dosage and the total dosage of the administered drugs;
many effective antibiotics for the treatment of urinary tract infections are nephrotoxic so that their use is limited to severe cases, such as chronic disease Intravesical administration of these drugs would prevent them from reaching the kidney; usually these drugs are administrated for several days, thus sustained release of drug into an infected bladder has an advantage; and
reduced systemic exposure enables the use of potent medicines that is restricted due to severe adverse effects, in a non-limiting example, the use of valrubicin to treat bladder cancer is restricted to patients for whom immediate cystectomy would be associated with unacceptable morbidity or mortality.

Furthermore, the present invention may significantly reduce the risk in using antibiotics by the reducing adverse effects and bacteria modification to be more antibiotics-resistant, thus enabling to broaden the indications for the treatment of a larger communities of patients. Several potent antibiotic drugs known to cause adverse reaction that limit their prescription to specific populations and specific clinical conditions. As non-limiting examples, such drugs include:
Amikacin is used for the initial treatment of serious gram-negative bacillary infections in hospitals where resistance to gentamicin and tobramycin have become significant problems, but its nephrotoxicity makes it dangerous to patients with impaired renal function and those that need prolonged therapy;
Plazomicin that is indicated by the FDA for complicated UTI for patients with no other alternative;
Cefiderocol that is indicated by the FDA for the treatment of complicated UTI including pyelonephritis caused by susceptible gram-negative bacteria in adults with limited alternative treatment options;
Quinolone and fluoroquinolone antibiotics that their use was restricted by EMA following a review due to the risk of long-lasting, disabling and potentially irreversible adverse reactions affecting multiple, body systems including musculoskeletal, nervous, psychiatric and senses);
Other antibiotic drugs used for various UTI conditions include: Amoxicillin, Ceftriaxone, Cephalexin, Ciprofloxacin, Fosfomycin, Levofloxacin, Nitrofurantoin, Sulfamethoxazole and trimethoprim combination (not suitable for patients with liver disease or folate deficiency), Imipenem (that affects people with brain disorders and colitis).

In other embodiments, the drug is an anti-sensation drug for the treatment of irritation within the bladder or interstitial cystitis. A non-limiting example of such a drug is lidocaine. In yet other embodiments, the drug is selected from the group of hormones when treating within the vagina body cavity delivering, for example progesterone or a similar active agent.

In yet other embodiments, the drug is selected from the group of antibiotics or other drugs for the treatment of urinary tract infection. Such drugs can be as non-limiting examples selected from the following list: nadofaragene firadenovec, enfortumab vedotin, sacituzumab govitecan, brilacidin and amikacin.

In other embodiments, the drug is botulinum toxin (Botox) for the treatment of overactive bladder causing stress incontinency. In other embodiments, the drug is from the family of clotrimazole, or other azoles, for example clotrimazole, for the treatment of antifungal spectrum for local treatment of fungal infections such as Candida.

In a further embodiment, the biodegradable and biocompatible article is a compressible sponge, and the method thus comprises an additional step of squeezing the sponge and pushing the sponge, while it is compressed, into the catheter prior to inserting the catheter into the body cavity.

In yet further embodiment, the biocompatible and biodegradable article is a collagen-containing sponge comprising an absorbable gelatine sponge, collagen, and an active ingredient. In other embodiment, the biocompatible and biodegradable article is a foamed absorbable polymeric matrix comprising non-absorbable, drug-carrying microspheres embedded in said matrix is suited for degradation inside the body cavity and release of the microspheres, which are eliminated through the body cavity. As a non-limiting example, this foamed absorbable polymeric matrix is composed of poly(D,L-lactide-co-glycolide)-co-polyethylene glycol di-block copolymer, said copolymer is used to impart a short degradation time to the article.

In yet further embodiment, the biocompatible and biodegradable article is a gelatine sponge. Degradation time of the collagen or the gelatine and other sponges can be controlled by the cross linking of the material.

In another embodiment, the biocompatible and biodegradable article is a microsponge or plurality of microsponges comprising porous, microscopic, polymer-based microspheres that are suited for suspending or entrapping the drug and releasing the drug in a sustained flow out of the microspheres. The biocompatible and biodegradable microspheres of this embodiment may be incorporated into a formulated product selected from a gel, cream, liquid, or powder. Treating prostate cancer by injecting micro-sponges loaded with drugs and gel into the proximity of the cancer bulk has the advantage of local administration of chemotherapy. As non-limited example chemotherapeutic drugs for the family of bulk cancer drugs, as well as hormones anti androgen drugs, may be used.

Suitable therapeutic agents include, but are not limited to, chemotherapeutic agents such as paclitaxel and rapamycin, immune checkpoint inhibitors such as anti-CTLA-4 antibodies, anti-PD-1 antibodies, anti-PD-L2 antibodies, and tyrosine kinase inhibitors. In some embodiments, the drug used in the drug delivery system of the present invention is a PD-1 inhibitor. PD-1 inhibitors are a class of drugs that block the activity of the PD-1 protein, which is found on the surface of T cells. PD-1 normally helps to regulate the immune system and prevent it from attacking healthy cells. However, in some cases, PD-1 can also prevent the immune system from attacking cancer cells. PD-1 inhibitors work by blocking the activity of PD-1, which allows the immune system to attack and kill cancer cells. The drug delivery system of the present invention is thus suitable for local administration of a PD-1 inhibitor for cancer treatment. The article (100) may also comprise a PD-1 inhibitor. Some non-limiting examples of PD-1 inhibitors include dostarlimab and pembrolizumab. These drugs are typically administered intravenously. However, the method of the present invention makes it possible to locally administer PD-1 inhibitors, which may help to reduce side effects and improve efficacy.

In certain embodiments, the drug delivery system utilizes a humanized monoclonal antibody (mAb) h11B6, which specifically targets human kallikrein 2 (hK2), a protein overexpressed in most prostate cancers. By binding to hK2, mAb h11B6 can induce prostate cancer cell death. This antibody is currently under investigation in a Phase 0 clinical trial for prostate cancer treatment.

The present invention provides a method for the localised delivery of mAb h11B6 directly into the prostate tissue. This approach may enhance efficacy and reduce systemic side effects by bypassing the prostate blood barrier (PBB), allowing the antibody to diffuse locally rather than relying on systemic circulation. The mAb h11B6 can be conjugated with a cytotoxic agent such as a radioactive material (e.g., iron-55, iodine-131) or another anti-cancer drug. Furthermore, these agents can be used in combination. For example, an anti-PD-1 antibody could be combined with a chemotherapeutic agent, or a tyrosine kinase inhibitor could be combined with h11B6 conjugated to a radioactive material. These agents, alone or in combination, can be delivered directly into the prostate using the methods described herein.

In another embodiment, the drug delivery system utilizes an anti-CTLA-4 monoclonal antibody. Anti-CTLA-4 antibodies block the activity of CTLA-4, a protein found on the surface of T cells that regulates immune responses. While CTLA-4 helps prevent the immune system from attacking healthy cells, it can also hinder the immune system's ability to target cancer cells. By blocking CTLA-4, these antibodies enhance the immune response against cancer. This drug delivery system is particularly suitable for intravesical administration of anti-CTLA-4 antibodies to treat bladder cancer.

Non-limiting examples of anti-CTLA-4 antibodies include ipilimumab and tremelimumab. While typically administered intravenously, the present invention enables localised administration, potentially reducing side effects and improving efficacy. In certain embodiments, anti-CTLA-4 antibodies are combined with an anti-PD-1 antibody, such as nivolumab, to further enhance the anti-cancer immune response.

Targeted drug delivery is a highly effective method for treating cancer. The present invention offers a novel approach for localised delivery to treat prostate cancer. Human kallikrein 2 (hK2), a protein largely confined to prostate tissue, is often overexpressed in prostate cancers, making it an ideal target. The humanized monoclonal antibody (mAb) h11B6 specifically binds to hK2 and has shown promise in early clinical trials (NCT04116164). While current methods involve intravenous administration, this invention proposes direct injection of the antibody into the prostate tumour.

This targeted approach aims to optimise drug distribution, ensuring that a sufficient concentration of the antibody reaches the tumour while minimizing exposure to surrounding healthy tissues and the rest of the body. By concentrating the treatment at the tumour site, the method of the present invention has the potential to improve efficacy and reduce side effects.

According to the method of the present invention, patients with advanced, localised prostate cancer receive a single injection of the therapeutic agent directly into the tumour. This is performed using ultrasound guidance to ensure accurate placement. The injection consists of:
(a) 2 mg of [111ln]-DOTA-h11B6 (a radiolabelled version of the antibody for tracking and potential therapeutic effect);
(b) optionally 8 mg of unlabelled h11B6 (to increase the overall concentration of the antibody);
(c) a specialized gel to control the release of the antibody over time; and
(d) potentially, micro-sponges to further enhance drug delivery.

This localised approach offers several advantages:
Reduced systemic exposure: By delivering the drug directly to the tumour, the rest of the body is spared from potential side effects.
Improved efficacy: Concentrating the drug at the tumour site may lead to better treatment outcomes.
Optimised pharmacokinetics: The gel and micro-sponges help to control drug release, ensuring a consistent therapeutic effect over time.

In yet another embodiment, the biocompatible and biodegradable article is a plurality of microsphere hydrogel sponges. In a non-limiting example, the hydrogel sponge comprises poly (trimethylolpropane ethoxylate triacrylate) microspheres cross-linked by a hydrogel, which is formed by a starch-based bifunctional emulsion stabiliser.

The biocompatible and biodegradable article of the present invention may be a marine sponge-derived natural sponge. In still another embodiment, the biocompatible and biodegradable article comprises a plurality of biodegradable nano-sponges, said nano-sponges are nanosized drug carriers with a three-dimensional structure created by crosslinking polymers, and they are suited for providing a controlled drug release pattern with targeted drug delivery. The biocompatible and biodegradable article may also comprise an enteric coating for the extended release of the drug.

Non-limiting examples of the materials used in the biocompatible and biodegradable article are chemical compounds selected from β-cyclodextrins, alginates, carboxymethyl cellulose, chitosan, carrageenans, cross-linked cellulose nanofibers, and collagen, gelatine or combinations thereof.

There are several known methods for preloading drug delivery systems with a drug. In one embodiment of the present invention, the biocompatible and biodegradable article is preloaded with the drug by a liquid-liquid suspension polymerisation or a quasi-emulsion solvent diffusion technique.

In one embodiment of the present invention, the biocompatible and biodegradable article is made of foam and is preloaded with the drug.

In one embodiment, the drug loaded into the sponge is in a liquid state. In other embodiments, the drug may be in various forms, including an emulsion, a supersaturated emulsion, a suspension, a powder, or an ointment. This versatility enables the delivery of drugs with limited water solubility, which can be incorporated into the sponge in a suspension or other suitable form.

According to another aspect, the present invention describes a preparation method for the biocompatible and biodegradable article of the present invention, wherein said method is selected from liquid-liquid suspension polymerisation, quasi-emulsion solvent diffusion, multiple-emulsion solvent diffusion, porogen addition method, lyophilisation, and ultrasound technique. These are known techniques in the art, and their disclosure is incorporated herein by reference.

The method and the system described can be used in any inner body cavity. A non-limiting example is a bladder. Other examples are renal pelvis of the kidney, cavity of lungs, GI tract, vagina, rectum, mouth, nasal system, and ears.

The methods and systems of the present invention are applicable to various tissues and organs within the body. The drug-loaded sponge, which may be in a squeezed or microspheric form, can be injected directly into the target tissue. Optionally, a gel can be incorporated to control the drug's diffusion rate. Non-limiting examples of suitable target tissues include the prostate, liver, and breast. Alternatively, the therapeutic agent can be pre-mixed with the gel and then injected into the target tissue. This approach provides controlled drug release from the gel matrix directly within the tissue.

As mentioned in the background part of the invention, in the realm of healthcare, managing urinary incontinence (UI) remains a significant challenge. Millions of people worldwide grapple with this condition, experiencing involuntary urine leakage that disrupts daily life and causes emotional distress. Existing solutions, while offering some relief, often fall short in user comfort, effectiveness, or targeted treatment. The present invention thus provides novel devices, systems, and methods to revolutionize UI management enabling individuals, specifically women (although also suitable for men), with UI to regain control, dignity, and a life free from leaks.

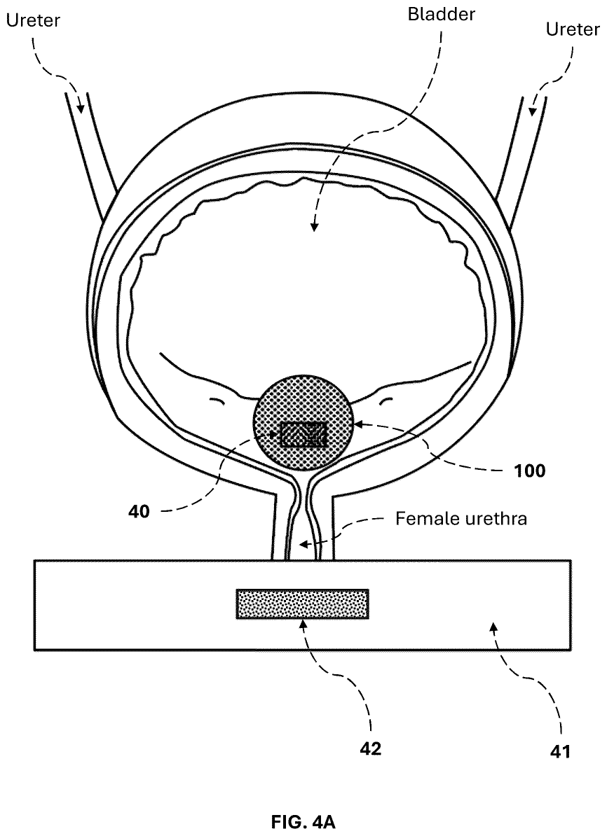

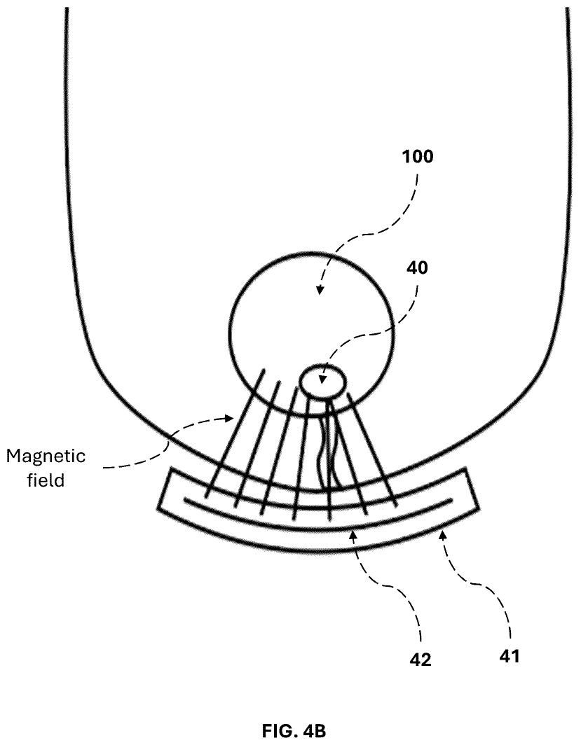

Reference is now made to FIG. 4A describing yet another aspect of the present invention, which is the treatment of urinary incontinency in a patient in need thereof. A deformable, biocompatible and biodegradable article, such as sponge (100), with a magnet (40), are placed within the catheter (shown in FIG. 1D, but not shown in this figure), and this sponge with the magnet are delivered into a women bladder partially filled with urine. A pad (41) with an external magnet (42) is placed in the woman perineum proximity, such as both magnets are in oppose direction, thus attracting each other. The sponge (100) may optionally incorporate a drug. Non-limiting examples of the drug is botulinum toxin, desensitisation drugs, such as lidocaine, chemical solvents, such as DMSO, or antibiotics.

Reference is now made to FIG. 48 illustrating the example of the pad (41) designed to be located in a woman's perineum, such that when the pad (41) with a magnet (42) is within proximity to the article (100) with an internal magnet or ferromagnetic element (40) in the bladder, an external magnetic field, which is created by the magnet (42) then attracts the ferromagnetic element (40), thereby pulling the article (100) towards the bladder's neck and closes the entrance of the urethra, the present invention provides a system for treating or preventing urinary incontinence of a patient, the system comprising: (a) an external part consisting essentially of a magnet or a ferromagnetic element (42) incorporated into a pad (41), and designed to be placed in proximity to an urethral orifice; and (b) a deformable, biocompatible and biodegradable article, such as sponge (100), comprising a ferromagnetic element, a magnet, or a paramagnetic element (40), wherein said article has an average molecular weight of about 1, such that the article (100) is buoyant in urine; and is designed to be inserted into the patient's urinary bladder, and wherein said system is designed and configured such that when the article (100) is disposed within said urinary bladder, a magnetic force from the external magnet (42) applied to said magnet (40) causes said article (100) to move towards said external part and thereby seal the patient's urethral orifice and prevent involuntary urine leakage.

The present invention further provides a system for treating or preventing urinary incontinence of a patient, the system comprising a deformable, biocompatible and biodegradable article, such as sponge (100) comprising a ferromagnetic element, a magnet, or paramagnetic element (40) wherein said article (100) has a molecular weight of about 1, such that the article (100) is buoyant in urine, and is designed to be inserted into the patient's bladder, wherein the system is designed to interact with an external magnet or a ferromagnetic element (42) incorporated into a pad (41) positioned outside the bladder for closing.

The present invention further provides a method of treating or preventing urinary incontinence of a patient, comprising the steps of: (a) providing a deformable, biocompatible and biodegradable article, such as sponge (100) comprising a ferromagnetic element, a magnet, or paramagnetic element (40) wherein said article (100) has a molecular weight of about 1, such that the article (100) is buoyant in urine; (b) placing said article inside a patient's bladder; and (c) placing near the urethral orifice an external part consisting essentially of a magnet or ferromagnetic element (42) incorporated into a pad (41), wherein: (i) when the external part is adjacent to the urethral orifice, a magnetic force from said magnet or ferromagnetic element (42) causes said article (100) with a magnet (40) to move towards the urethral orifice (bladder's neck or urethra's neck) and thereby close/seal the patient's urethral orifice and prevent involuntary urine leakage; and (ii) when the external part is distanced from the urethral orifice (e.g. when removing the underwear holding the external part), the magnetic force is reduced thereby causing said article (100) to move away from the urethral orifice and thereby allow urine to exit.

The term “urethral orifice” as used herein refers to the women's perineum or vaginal opening or a male's perineum. The term “magnet” may refer to a permanent magnet that is a ferromagnetic material such as iron that was magnetized to become a magnet. The term “paramagnetic material” refers to materials that can be attracted by a magnet.

In specific embodiments thereof, the article (100) of the invention, comprising a ferromagnetic element, a magnet, or a paramagnetic element (40), is water-sealed. In certain embodiments of the system according to any of the embodiments above, this article includes a material having a specific gravity that is less than a specific gravity of water, said material allowing for the article (100) to be buoyant in aqueous fluids such as foam, porous solid, liquid and gaseous materials. In certain embodiments of the system according to any of the embodiments above, article (100) is free-floating in said urinary bladder.

In certain embodiments of the system according to any of the embodiments above, the article (100) is a buoyant so that it floats in the urine in the bladder, and has a specific molecular weight close to 1 (like water), namely from 0.85 to 1.2, such as 0.9, 0.95, 1, 1.05, 1.1, and 1.15, so that it will not bounce within the bladder and will not hit the balder wall.

In certain embodiments, the system according to any of the embodiments above further comprises an applicator for placing said article (100) in the patient's bladder. In certain embodiments described below, the system of the invention further comprises a flexible funnel holding said article (100), said funnel comprises a distal end designed to be positioned in the patient's urethra, and a proximal end designed to reside within the urinary bladder at the urethral orifice, wherein said funnel comprises a mesh at its broad part designed to prevent the article (100) from escaping from the urethral orifice and maintain it close to the urethral orifice to enable the creation of a magnetic pull between the magnets (40) and (42), i.e. internal and external magnets.

In certain embodiments, the above system further comprises an applicator for placing said funnel holding said article (100) in the patient's bladder and urethra. In certain embodiments of the system according to any of the embodiments above, the flexible funnel has an expandable tip, such as a disk or a malecot-flower, designed to expand within the urethra to therefore anchor said funnel in place and verify that the broad part of the funnel is secured against the tissue at the urethral orifice and verify that the article (100) is sufficiently close to the urethral orifice to enable the creation of a magnetic force between the article (100) and the external part.

In certain embodiments of the system according to any of the embodiments above, the external part is: (i) part of an absorbing pad (41); (ii) a part of or is designed to be inserted in or be a part of the patient's underwear; (iii) consists essentially of a magnet and said article (100) comprising a ferromagnetic element (40); (iv) consists essentially of a ferromagnetic element (40) and said article (100) comprising an additional magnet, or (v) consists essentially of multiple beads made of paramagnetic small balls.

In certain embodiments, the system according to any of the embodiments above further comprises: (i) a material having a specific gravity that is less than a specific gravity of water, such as closed-cells foam/sponge, porous solid, liquid and gaseous materials, said material allowing for the article (100) to be buoyant in aqueous fluids; and/or (ii) a flexible funnel holding said article (100), the funnel comprises a distal end designed to be positioned in the patient's urethra, and a proximal end designed to reside within the urinary bladder at the urethral orifice, wherein said funnel comprises a mesh at its broad part designed to prevent the article (100) from distancing from the urethral orifice and maintain it close to the urethral orifice to enable the creation of a magnetic pull between said ferromagnetic element (40) and said magnet (42), i.e. internal and external magnets.

In certain embodiments of the system according to any of the embodiments above, the flexible funnel has an expandable tip, such as a disk or a malecot-flower, designed to expand within the urethra to therefore anchor said funnel in place. Notably, any other mechanism that is suitable for anchoring the funnel in place can be used.

It should be noted that the features and components of the devices and systems according to any of the embodiments above are intertwined and can be used in any device or system according to the invention and can be used in any method of the invention.

The invention will now be illustrated by the following non-limiting examples and by reference to the accompanying drawings which are to be considered only as representative examples of possible embodiments of packages of the invention.

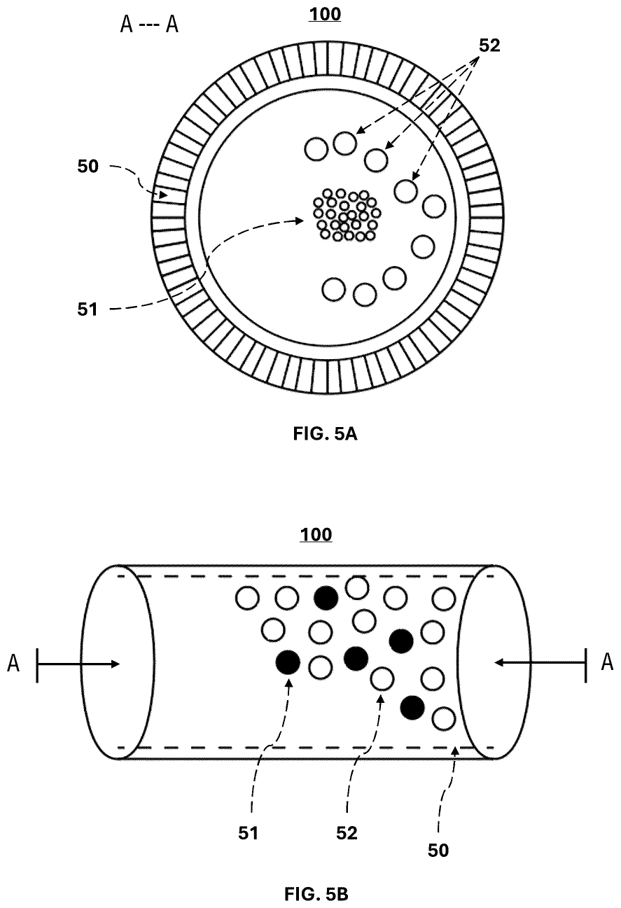

FIG. 5A describes one embodiment of the article (100) of the invention, comprising numerous ferromagnetic or paramagnetic beads (51) within it, which are equivalent to the internal magnet (40) mentioned above. Also illustrated is a soft cushion (50) surrounding the article (100) to prevent the risk of causing damage to the tissue and to enable adequate fitting to the bladder's opening/neck for better sealing thereof. The article (100) is buoyant so that it floats in the urine in the bladder. As such, to reduce the specific weight thereof to be close to 1 (like water), e.g., 0.85, 0.9, 0.95, 1, 1.05, 1.1, 1.15, 1.2, it contains material that is lighter than urine, such as air, air-filled sponge, or beads filled with air (52). Illustrated are beads (52) made of babbles full of air or close cell sponges. Thus, the specific weight of the article (100) is significantly reduced. This is to avoid the bouncing of the article (100) and hitting the bladder wall.

The optimum specific weight of the article (100) should be slightly above 1, and preferably 1.1. Since the specific weight of the ferromagnetic beads is close to 8, there is a need to insert about 8 times more, volume-wise, the number of beads (53). Notably, since the specific weight of the article (100) is little above 1 g/cm², it does not bounce within the bladder, but rather normally located at its bottom—in close proximity to the perineum.

The volume of the article (100) may contain some water or gel. The diameter size of the article (100), when within the bladder, is about 2 cm. But in other embodiments, it can be 4 cm, 3 cm, 5 cm, and up to 7 cm. For insertion of the article (100) to the bladder, it can be squeezed to a cylindrical shape as shown in FIGS. 1A and SB to allow easier insertion through the urethra.

A preferred diameter of the article (100) when squeezed is about 4 to 5 mm, but it can be less or as large as 8 mm or more. For example, when the article (100) is 4 cm in diameter, its volume is 33 cm³, and in its cylindrical shape of 6 mm diameter its length will be 11.6 cm. In another embodiment, when the diameter of article (100) is 2 cm, its volume is about 4 cm³, and thus, when in a cylindrical shape of 6 mm diameter during insertion, its length is about 14 cm. In another embodiment, the article (100) contains a gel as well to make it soft enough and flexible enough thus considerably improving its sealing capabilities of the bladder's opening/neck.

In certain embodiment, the ferromagnetic or paramagnetic beads (51) are made of ferromagnetic material. In such a case, the polarity direction of the magnet (42) within the pad (41) is of importance. Since in one polarity direction, it will attract the article (100) while directing the external magnet (42) to the other direction it will push the article (100) away. In such an example, the soft cushion (50) is placed only on one side of the article (100). In one embodiment, the ferromagnetic or paramagnetic beads (51) are made of a paramagnetic material such as iron. In such a case, the polarity direction of the magnet (42) within the pad (41) is of no importance. In such an example, the cushion (50) may be placed all around the article (100) as shown in FIG. SA and explained above.

Reference is now made to FIG. 5B showing the article (100) in a compressed mode, which results in a cylindrical shape having a diameter of about 5 mm so as to enable pushing it into the patient's bladder using the insertion aid (104), for example through a regular catheter.

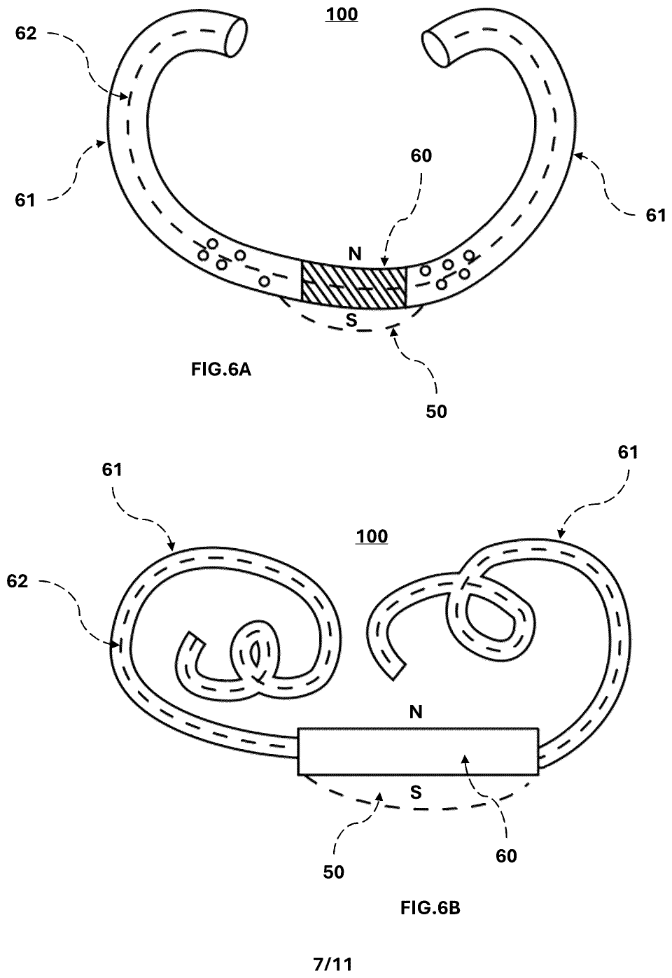

FIGS. 6A and 68 are illustrations of alternative configurations of the article (100) of the present invention used for the treatment of urinary incontinency in a patient. Reference is first made to FIG. 6A showing the article (100) comprising the magnet bar (60) having one side north (N) pole and another side south (5) pole. A tubular cylindrical-shaped close-cell sponge (61) is attached to both sides of the magnet bar (60). A pre-shaped memory nitinol wire (62) is located within the article (100), having a pre-shape designed to produce a desired geometry. Also illustrated is a soft cushion (50) located at the bottom of the magnet (60) to improve the sealing of the bladder's neck and the external urethral orifice when the article (100) is attracted by the external magnet (42). FIG. 6B illustrates the article (100) when it is delivered into the bladder. The shaped memory nitinol wire (62) gives the article (100) a shape that is more adopted to the bladder anatomy. This is one, non-limiting example, of a possible shape that can be used.

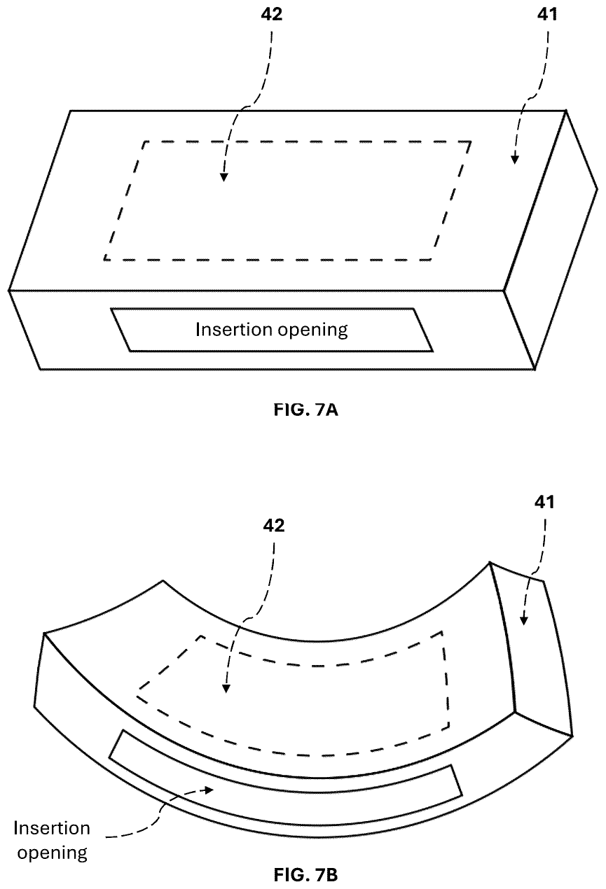

FIGS. 7A and 7B schematically show the external part of the system of the present invention used for the treatment of urinary incontinence. FIG. 7A illustrates the embodiment when the external magnet (42) seen in FIG. 4A of the system of the invention is held within a dedicated absorbent pad (41) as commonly used by females. When the magnet (42) is then brought close to perineum, it attracts the paramagnetic beads (51) seen in FIG. SA, or the ferromagnetic element (40) seen in FIG. 4A, thereby causing the soft cushion (50) to seal the opening of the urethra.

Reference is now made to FIG. 7B showing the external magnet (42) made of a flexible, bendable magnetic material so that it can have a better fit when used in the pad. For example, a banana-shaped plate magnet can be used. In specific embodiments, the magnet (42) can be reused by pulling it out of the pad (40) and re-inserting it into a dedicated pocket in a new pad (40) as shown in FIG. 7B. The magnet (42) may be a flexible or bendable magnet. The ability of magnet's bending increases the effectiveness of its ability to attract the article (100) of the invention as seen in FIG. 7B, where the geometry of the field lines helps centralize the article (100) in the bladder neck. Another advantage is that the curved or bent shape is better adapted to the user's anatomy.

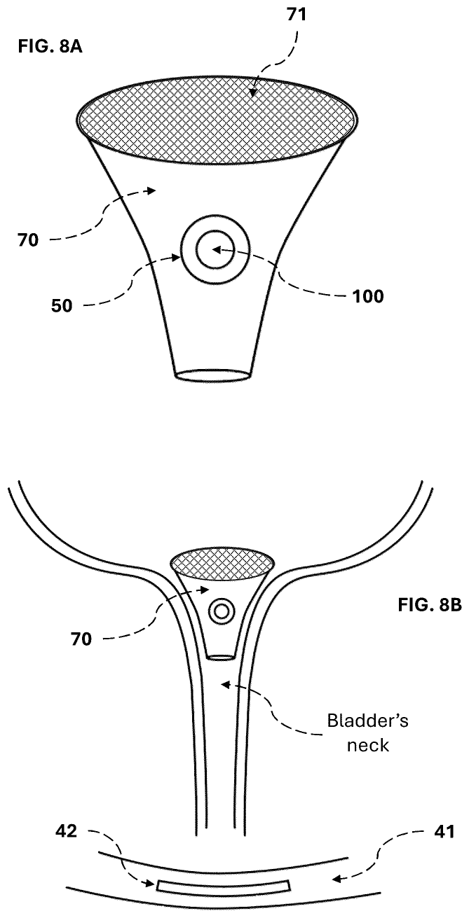

Reference is now made to FIGS. 8A and 8B illustrating an alternative embodiment of the invention, in which the article (100) of the present invention is held within a dedicated funnel (70). This configuration allows making sure that the article (100) is always in close proximity to or in the bladder's neck. As seen in these figures, the article (100) can be folded to enable easier insertion through the urethra and into the bladder. The article (100) is located within the assembly and is covered with a soft cushion-like element (50). The broad side of the funnel (70) is covered with a screen or mesh (71) to ensure the article (100) stays within the assembly and does not float too far away from the bladder's neck.

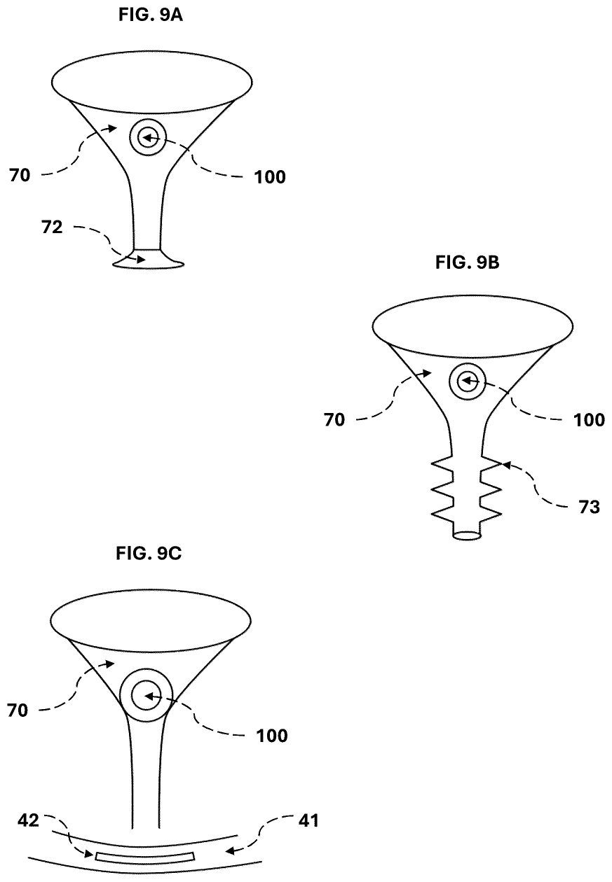

Reference is now made to FIGS. 9A and 9B schematically showing two types of funnel structures. FIG. 9A illustrates a funnel structure (70) with a disk portion at its narrow side, and FIG. 9B illustrates a funnel structure (70) with a malecot-flower at its narrow side. In certain embodiments, as illustrated in these figures, to ensure that the article (100) will stay in place, the funnel (70) has a bendable protrusion (72) at its proximal (narrow) side. The bendable protrusion (72) can be in any shape and size, such as disk-shaped or malecot-flower-shaped protrusion (73). Alternatively, or in addition, the assembly (or its narrow side) can be made of plastic that is normally expandable, or can be made of metal like nitinol that is memory shaped to be open. FIG. 9C is an illustration of how the article (100) of the invention closes the funnel when a magnetic force is applied. As seen in this figure, when a pad (41) holding a magnet (42) is placed in/on the perineum, it attracts the article (100) that then seals the funnel (70) and prevent urine from exiting.

To sum up, the present invention relates to the method described above for the treatment of urinary incontinence by placing a magnetic soft buoy stopcock inside a bladder of a patient that provides additional barrier against urine leak, allows for normal voiding, and can be simply placed and/or removed. This magnetic stopcock comprises a deformable, biocompatible and biodegradable article of the present invention, preferably in a form of a closed-cell squeezable sponge, where magnet, magnetic or ferromagnetic particles are placed inside the sponge. An external pad contains a set of magnets and is designed to be worn by a woman, placed against her perineum. Said magnets are designed to provide a strong attraction of the stopcock. The shape of the sponge fits the shape of the ureteral neck and the positioning of the magnetic or ferromagnetic particles is designed to direct the stopcock into the ureteral opening against the external magnetic pad. The sponge article may have both an open-cell sponge part and a closed-cell sponge part that is filled with air. Such article designed to have a specific weight close to one has an essential advantage of preventing bouncing in the bladder, while avoiding the sponge being stuck in the bladder neck, hence interrupting natural voiding.

Toward insertion, the stopcock is squeezed and placed in an insertion aid of the invention and is pushed into the woman's bladder. After being placed inside the bladder, the stopcock is either pulled by the pad magnets to close the ureteral opening, or when the pad is removed, it buoys up from the closed position inside the bladder neck, allowing for normal voiding.

A retrieval device comprises a magnet and a collapsible grip mechanism. The grip mechanism is collapsed when the retrieval device is inserted into the bladder. Post insertion, the grip mechanism is opened, the stopcock is magnetically attracted to the retrieval device, with no need for visual aids, and the grip mechanism is closed to attach and compress the stopcock and enable its removal from the bladder. The retrieval device enables the replacement of the buoy stopcock either periodically or for safety.

In a non-limiting example, the act of locating and attaching the buoy stopcock to the said retrieval device can be externally verified either electronically or magnetically. The magnets in the pad and or the sponge are made of neodymium or samarium cobalt, that provide strong magnetic force.

The sponge closed-cells design and the magnetic and/or ferromagnetic particles, are designed to provide specific gravity close to one for optimal comfort: a) reduce the bouncing effect when the pad is removed (that could occur if the specific gravity is too low); and b) prevent hitting the bladder wall while moving (if its weight is too high).

In one embodiment, the sponge incorporates magnetic or ferromagnetic beads or particles, while the magnet is housed exclusively within the external pad. This configuration offers the advantage of using smaller, more easily inserted spheres within the bladder. The magnetic force in this case originates solely from the magnet located in the pad.

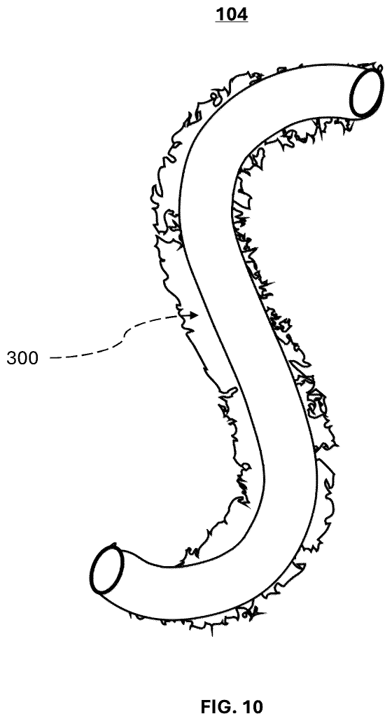

FIG. 10 describes yet another embodiment of an urethral catheter, usually known as a “double J” or “pigtail” catheter, covered with a coating layer (300). This catheter consists of a preloaded sponge with drug and gel as described. The coating layer (300) may be made of a sponge material that can hold a drug. This embodiment teaches how to sustain release of the drug into the ureter surface for treating ureter cancer cells.

In a specific embodiment, the treatment method of the present invention is intravesical. As such, it is not a systemic treatment, and it allows to reduce the total dosage the whole body is exposed to while keeping the same amount of a drug in the target, treated area. For example, the present method allows to treat the bladder with a drug having minimum toxicity to kidneys.

One example of the practical application of the method of the present invention is the treatment of bladder cancer. Most anti-cancer drugs are most effective only during some short period of the cancer cell's life cycle. Basically, they are more susceptible during cell division. When a chemotherapy drug is delivered to a patient's body and remains there for a short period of time, it will only encounter a few cells during the receptive period. In the case of bladder cancer, cell proliferation usually begins as small polyps and small spots on the inner wall of the bladder. If detected early, resections are performed followed by a series of bladder washes with immunotherapy drugs such as Bacillus Calmette-Guérin (BCG) and anti-cancer drugs such as mitomycin C. When a chemotherapy drug is used, the longer the drug remains in the bladder, the more effective the treatment. Thus, the longer drug exposure provided by the method of the present invention is a great advantage, leading to much more effective anti-cancer treatment. For example, mitomycin C is known to be an effective drug for superficial bladder cancer, and the suggested method of this invention can be used for more deep bulk cancer.

One additional example of the practical application of the method of the present invention relates to the treatment of bladder cancer. The present invention teaches a method to perform a chemoablation of tumour bulk sites that can serve as an alternative treatment to tumour resections. Chemoablation of solid tumours is instigated with prolonged slow-release of chemotherapy from a gel-mixture that is inserted into the urinary tract. This method is disclosed in patent documents by the present inventors: U.S. Pat. Nos. 9,540,407, 9,801,854, 9,884,028 and more. That method requires a strong match of the gel-carrier properties to the chemotherapy agent physical and chemical properties, such as solubility. Thus, the method is limited to a narrow range of combinations of drugs, specific drug mixes and drug dwell duration. The present invention enables the use of any chemotherapy agent, for any concentration, for any dwell time—enormously widening the range of clinical options and improving the efficacy.

Another example of the practical application of the method of the present invention is the treatment of urinary tract infection (UTI). There are different categories of patients suffering from UTIs: with a mild single infection, some of whom are treated with antibiotics, high-risk patients receiving antibiotics prophylactically, for example, patients with an urethral catheter, patients with a low immune status, or patients with HIV, and patients with a chronic infection, when these patients must complete a six-month course of antibiotics, with a high rate of comorbidities and very low success rates. The method according to the present invention is very effective in the treatment of the first two categories of patients, where the ability to provide a long antibiotic effect is very important.

Another example of the practical application of the method of the present invention is the treatment of overactive bladder. Many women suffer from stress incontinence, where they pass a few drops of urine when climbing stairs, jumping, or sneezing. Over active bladder and Stress urinary incontinence occurs due to uncontrolled bladder contractions that are stimulated by certain nerves. Medical treatment today is usually done through muscle relaxation regimens. One of them is, for example, an injection of botulinum toxin into the wall of the bladder to treat stress urinary incontinence. This treatment has its own side effects and serious adherence problems, and its effectiveness is limited to about six months. Treatment according to the method of the present invention may use botulinum toxin (Botox) and the like by slow release over a long period of time to effectively treat stress urinary incontinence. An organic solvent such as DMSO can be used to increase the permeability of Botox through the urothelium. Thus, the present invention describes the treatment of such patients with Botox where the Botox is released over a long period of time paralysing the muscles that cause the bladder uncontrolled contractions.

The advantages of local treatment of bladder diseases over systemic administration are delivery directly to where it is needed, effects on other organs, minimization of drug exposure to the kidneys, which minimizes nephrotoxicity and possible lever damage, reduces the total amount of the drug necessary to achieve therapeutic levels, reduce side effects and adverse effects, the amount of drug used is well below the permitted peak level, which allows the use of a much higher concentration of the drug, but is limited to the bladder area. Even if some of the drugs reach the tissue of the bladder wall, they still need to pass through the urothelium to reach the inside of the bladder. In some indications, such as the UTI, where the biofilm adheres to the inner wall of the bladder, or superficial cancer, where cancer cells are located on the surface of the inner wall of the bladder, current systemic administration is most ineffective.

With regard to the ineffectiveness of systemic administration to the bladder, it should be additionally noted that in the treatment of chronic UTIs, colonies of bacterial foci are formed on the internal urothelium of the bladder. It is very difficult to supply antibiotics to treat this biofilm of colonies. The urothelium is impervious and limits the passage of the antibiotic agent through it from both sides. This means that only a small amount of an antibiotic agent that is administered systemically, reaches the inner bladder wall, where the biofilm of bacterial colonies is located. The method of the present invention overcomes this problem by non-systemic antibiotics drug delivery, and direct placement on top of the colonies.

In addition, many of the drugs used are known to be toxic to the kidneys. Nephrotoxicity limits the use of these drugs when administered systemically. Bearing in mind that these drugs, when administered systemically, must reach the kidneys. However, with intracavitary or intraluminal administration, only a very low dose of the drug is required, and of this smaller amount, only very few reach the system, while the lion's share is excreted in the urine without reaching the kidneys. The method of the present invention also overcomes this problem by the non-systemic drug delivery and extended release of a controlled amount of the drug inside the bladder. In addition, a DMSO solution may be used in conjunction with or as a pre-treatment, in order to ‘break’ the biofilm, thus allowing even more effective treatment of the antibiotic drug. The drugs, which are most effective to treat UTI with high nephrotoxicity that can be enabled to use when giving locally instead of systemic. The present invention enables the use of certain drugs that cannot or very rarely be used due to their nephrotoxicity.

The ability to administer the drug directly to the place where it is needed and for a long period of time enable reducing the total doze required. For example, when using antibiotics usually 10 percent of the systemic doze is practically reaching the bladder used, and in some embodiment even as low as 1 percent. Also, the peak doze is much less than the systemic administration thus the expected side effects and adverse effects are expected to be much lower.

In a particular embodiment, the drugs used in the method of the present invention are selected from the following classes of drugs: antineoplastic drugs, chemotherapeutic agents, anti-infective agents (such as antimicrobial drugs, antiparasitic agents, antivirals), genito-urinary system drugs, anti-inflammatory drugs, analgesics, musculoskeletal system acting drugs, drugs acting on the blood and blood forming organs (such as antihemorrhagics, antithrombotic agents, antianemia drugs), dermatologic drugs (such as antifungals, antiseptic), gastrointestinal system (such as anti-obesity, acid-related disorders), metabolism drugs, neurological drugs, respiratory drugs including nasal drugs, cardio-vascular drugs, ontological drugs, corticosteroids drugs, analgesics drugs, antiparasitic drugs and anaesthetic drugs.

Non-limiting examples of the drugs used in the method of the present invention are amoxicillin, ceftriaxone, cephalexin, ciprofloxacin, fosfomycin, levofloxacin, amikacin, piperacillin, nitrofurantoin, trimethoprim, sulfamethoxazole, gentamicin, imipenem, meropenem, ceftolozane, cefiderocol, plazomicin, neomycin, kanamycin, paromomycin, bacitracin, vancomycin, colistin, polymyxin, amphotericin, quinolone, fluoroquinolone, nadofaragene firadenovec, enfortumab vedotin, sacituzumab govitecan and brilacidin.

In a particular embodiment, the gel described is mixed with a therapeutic agent and both are injected into a body tissue. The drug is eluted from the biodegradable reverse gelation gel for a long period of time in the area that it was injected. Such a treatment is very effective when fighting against local focal cancer, like prostate cancer. There is only little exposure to the whole body, and the process assures the release of chemotherapy for a long period of time at the tumour proximity.

The injection into the treatment zone can be performed using any available needle-based injection system, guided by imaging techniques such as ultrasound or MRI. For brain tumour treatment, guidance may be provided by real-time or previously acquired MRI, or by stereotactic systems.

In another embodiment, kidney cancer can be treated by delivering drug-loaded sponges, optionally with a diffusion-controlling gel, into the renal pelvis. This can be achieved through various approaches, including nephrostomy, where a catheter is inserted using a trocar system, endoscope, or a specialized catheter like the Melancon catheter. Other suitable injection devices known in the art may also be employed.

In a further embodiment of the invention, a kit for use in an intracavitary or intraluminal application and sustained release of a drug into the body cavity of a patient comprises the first (drug delivery) system of the invention, and instructions for use of said drug delivery system. In still another embodiment, a kit for use in treating or preventing urinary incontinence of a patient, comprising the second system of the invention, and instructions for use of said system.

In addition, the kits of the present invention comprise instructions in a form of an instruction manual or instruction sheet, which is generally written instructions, although an electronic storage medium, such as an optical disc, a flash drive or a link to a cloud containing the instructions, is also acceptable.

The kits of the invention may further include a label or package insert on or associated with a container containing a drug. The term “instruction sheet” is used to refer to the instructions included in the commercial packaging of therapeutic products as usual, which contain information about the indications, usage, dosage, administration, contraindications and/or warnings of the use of such therapeutic products. Suitable containers for drugs and items of the system include, for example, bottles, vials, syringes, blister packs, and the like. The container can be formed of various materials such as glass or plastic. The container may contain a drug, its pre-mix with a gel, or its formulation effective in treating the condition and may have a sterile access port. For example, the container may be an intravenous solution bag or a vial with a stopper pierceable by a hypodermic injection needle. At least one active agent in the composition is a drug used with the article of the present invention.

The label or package insert indicates that the composition is used to treat the selected condition, such as kidney cancer or urinary inconsistence. In addition, the label or package insert may indicate that the patient to be treated is a person with a condition such as a hyperproliferative disorder, neurodegeneration, cardiac hypertrophy, pain, migraine, or neurotraumatic disease or event. In one embodiment, the label or package insert indicates that the composition containing the drug can be used to treat conditions caused by abnormal cell growth. The label or package insert may also indicate that the composition can be used to treat other conditions.

Non-limiting examples of the drugs used in the manufacturing of the kits of the present invention are amoxicillin, ceftriaxone, cephalexin, ciprofloxacin, fosfomycin, levofloxacin, amikacin, piperacillin, nitrofurantoin, trimethoprim, sulfamethoxazole, gentamicin, imipenem, meropenem, ceftolozane, cefiderocol, plazomicin, neomycin, kanamycin, paromomycin, bacitracin, vancomycin, colistin, polymyxin, amphotericin, quinolone and fluoroquinolone.

The kit of the invention may further include other substances required from the viewpoint of medical business and users, including buffers, diluents, filters, catheters, needles and syringes.

In summary, the present invention provides a significant advancement in the treatment of localised prostate cancer. This method offers a more targeted and effective approach with potentially fewer side effects. By directly injecting therapeutic agents into the prostate tissue, the invention overcomes the limitations of the prostate blood barrier (PBB), which hinders the delivery of systemically administered drugs. This localised delivery strategy bypasses the PBB, enabling higher drug concentrations to reach the tumour.

### CONCLUSIONS

### Advantages of the Drug Delivery System and Method

The drug delivery system and method of the present invention offer a significant advantage in the treatment of various conditions, particularly cancers, by allowing sustained exposure of affected cells to therapeutic agents. This prolonged exposure is especially crucial for drugs that are most effective during specific phases of the cell cycle, such as during cell division. By maintaining a consistent drug presence, the device enhances the therapeutic effect and improves treatment outcomes.

One key advantage is in the treatment of bladder cancer. Most anti-cancer drugs, including those used in bladder cancer treatment, are most effective during cell division. Traditional drug delivery methods often result in short exposure times, limiting the number of cancer cells exposed to the drug during their vulnerable phase. The present invention overcomes this limitation by providing extended drug exposure within the bladder, leading to more effective outcomes.

Specifically, while conventional anti-cancer drugs like mitomycin C are known to be effective against superficial bladder cancer, the drug delivery system of the present invention can be used for deeper and bulkier tumours. Furthermore, this invention offers an improved approach for chemoablation of tumour bulk sites, providing an alternative to tumour resection.

Previous chemoablation methods rely on a gel mixture for the slow release of chemotherapy within the urinary tract. However, these methods are constrained by the need for precise matching of the gel-carrier properties to the specific chemotherapy agent's physical and chemical properties (e.g., solubility), thus limiting the range of usable drugs, concentrations, and dwell times. The present invention overcomes these limitations, enabling the use of a broader range of drugs, including the non-limiting examples of immune checkpoint inhibitors, such as anti-CTLA-4 antibodies, anti-PD-1 antibodies, anti-PD-L2 antibodies, tyrosine kinase inhibitors, and humanized monoclonal antibodies (mAb) h11B6, alone or in combination thereof, at any concentration and for any dwell time, dramatically expanding treatment options and improving efficacy.

### Addressing Nephrotoxldty

Another critical advantage of the present invention is its ability to address nephrotoxicity, a common side effect of many drugs, especially those used in cancer treatment. Nephrotoxicity refers to damage to the kidneys caused by the toxic effects of certain medications.

Traditional systemic drug administration, where the drug is distributed throughout the body, often leads to significant exposure of the kidneys to the drug, increasing the risk of nephrotoxicity. This can limit the use of potentially effective drugs or require dose reductions, compromising treatment efficacy.

The present invention overcomes this challenge by enabling localised drug delivery directly to the affected site, minimising systemic exposure and reducing the burden on the kidneys. This is particularly important for drugs with known nephrotoxicity, as it allows for their use at effective concentrations while minimising the risk of kidney damage.

For example, in the treatment of bladder cancer, the drug delivery system can be used to deliver chemotherapy drugs directly to the bladder, where they are needed, without exposing the kidneys to high drug levels. This localised approach allows for the use of potent anti-cancer agents that might otherwise be contraindicated due to their nephrotoxicity.

In addition, the present drug delivery system's ability to provide extended drug release further reduces the risk of nephrotoxicity. By maintaining a consistent drug concentration at the target site, the need for frequent high-dose administrations is eliminated, reducing the overall drug exposure to the kidneys.

In a specific embodiment of the invention, the drug delivery system is designed to further minimise nephrotoxicity by incorporating a biocompatible and biodegradable coating on the drug-carrying article. This coating acts as a barrier, controlling the release of the drug and preventing its rapid diffusion into the systemic circulation.

The coating can be made from various materials, such as polymers or hydrogels, that are designed to degrade slowly over time. This controlled degradation ensures that the drug is released gradually, maintaining a therapeutic concentration at the target site while minimising systemic exposure and the risk of nephrotoxicity.

Furthermore, the coating can be tailored to the specific drug being used, optimising its release profile and further reducing the risk of nephrotoxicity. For example, the coating can be designed to release the drug more slowly in the initial stages, reducing the peak drug concentration and its potential impact on the kidneys. This embodiment demonstrates the versatility of the drug delivery system and its ability to be adapted to address specific challenges, such as nephrotoxicity, associated with different drugs and treatment regimens.

By minimising nephrotoxicity, the present invention expands the range of therapeutic options available for patients, particularly those with cancers or other conditions that require potent medications with known nephrotoxic effects. It allows for the use of effective drug concentrations while minimising the risk of kidney damage, improving treatment outcomes and quality of life for patients.

### EXAMPLES

### Example 1: DruA Delivery System

The described invention is a controlled drug delivery system intended for use in the bladder, utilising a sponge embedded with a gel containing a drug and coated with an impermeable layer with one or more perforations. This system ensures a controlled release of the drug over a period of up to 5 days. The gel is formulated to possess specific rheological properties, including thermoreversibility, to facilitate its administration and functionality within the body.

### Gel Composition

All components used in the gel formulation must be approved for medical applications to ensure safety and compatibility.
Thermoreversible Agent: Compounds like Pluronic F-127 (an A-B-A type triblock copolymer) exhibit thermoreversible properties, allowing the gel to transition from a liquid at low temperatures to a gel at body temperature.
Viscosity Modifier and Stability Provider: Compounds such as Hydroxypropyl Methylcellulose (HPMC), Carboxymethylcellulose (CMC), Xanthan Gum and others may be used to modify the viscosity and provide stability to the gel.
Plasticiser: Compounds like Polyethylene Glycol 500 (PEG 500) and other PEG compounds act as plasticisers and help control the release rate of the drug.
Humectant and Stabiliser: Compounds such as Glycerol serve as humectants and stabilisers, maintaining the gel's consistency.
Solvent: Double Distilled Water (DDW) is used as the primary solvent for the gel components.
Drug: The gel can incorporate drugs for the treatment of bladder cancer. These agents, alone or in combination, can be delivered directly into the prostate using the drug delivery system described herein.

### Concentration Ranges

The gel's physicochemical properties may be modulated by modifying the concentrations of its different components within certain ranges. For example:
Thermoreversible Agent (Pluronic F-127): 15-30% w/w
Viscosity Modifier and Stability Provider (for example, HPMC): 0.1-1% w/w
Plasticiser (for example, PEG 500): 0.5-2% w/w
Humectant and Stabiliser (for example, Glycerol): 0.01-0.1% w/w
Solvent (DDW): Balance to 100%

### Properties and Functions

Thermoreversibility: Pluronic F-127 exhibits thermoreversible properties, allowing the gel to be administered in a liquid form at lower temperatures and subsequently form a gel at physiological temperatures (˜37° C.). This property is critical for ease of administration through a catheter and ensuring the gel remains in place within the bladder.

Controlled Release: The gel matrix controls the release of the embedded drug at a rate that maintains the drug concentration within therapeutic levels but below toxicity levels. This ensures effective treatment while minimising side effects.

### Sponge Characteristics

Material: The sponge used in this system must be biocompatible and biodegradable. Suitable materials include:
Collagen: A natural, biodegradable polymer.
Other suitable biodegradable polymers include gelatin, polylactic acid (PLA), polyglycolic acid (PGA), and their copolymers (PLGA). A collagen-gelatin matrix is particularly attractive for controlling the biodegradation rate while maintaining structural integrity during the degradation process.
The sponge coating may be composed of the same material as the sponge itself (for example, collagen) or a different material that tightly adheres to the sponge.

Function: The sponge is designed to be inserted into the bladder through a catheter, where it expands as the gel is injected into it. It ensures a controlled and sustained release of the drug as the bladder fills and empties. The gel composition, the sponge's physicochemical properties, the coating characteristics and perforations and the drug's nature and concentration determine the drug's release profile, release duration and resulting drug concentration in the urine.

### Gel Preparation

### Materials:

DW: 763.7 mL
PEG 500: 9.0 g (0.9%)
Glycerol: 0.5 g (0.05%)
HPMC: 1.8 g (0.18%)
Pluronic F-127: 225.0 g (22.5%)

### Procedure:

1. Pour the DW into a 2-L beaker and place it in an ice bath under an overhead stirrer.
2. Stir at medium speed (250 rpm) and add PEG 500 and glycerol, continuing for 5 minutes.
3. Increase speed to 800 rpm and gradually add HPMC over 8-10 minutes.
4. Adjust stirrer speed to manage foam formation, if necessary.
5. Ensure the solution temperature is below 8° C. before adding Pluronic F-127 gradually over 1 hour.
6. Homogenize the mixture for 10 minutes using an Ultra Turrax homogeniser, managing foam as needed.
7. Bottle the solution into a sterilised 2-L flask and refrigerate at 4° C. for 3-4 days.

### Testing Parameters:

Short term: Appearance, pH, gel point, viscosity (at the range of 4-40° C.).
Long term: Stability and biological contamination.

### Example Results

Appearance: Stable, homogeneous, colorless, odorless, transparent gel.
pH: 8.9 at 8° C.
Gel Point: 19.6° C.
Viscosity: Measured across a range of temperatures, indicating a significant increase as temperature approaches gel point.
   Packing: The gel is liquefied at 4° C. and bottled in sterilised containers for delivery, to be kept refrigerated at 4-8° C.

### Example 2: Gel in the Drug Delivery System for the Treatment of Upper Tract Carcinoma

In one of the aspects of the present invention, the drug delivery system of the invention is delivered to a kidney of a patient to treat upper tract carcinoma. The biocompatible and biodegradable article of the invention is pre-loaded with an anti-cancer drug that is delivered to one or more places within the renal pelvis of a kidney. The biocompatible and biodegradable article then can release the drug for several days.

Placement of the biocompatible and biodegradable article, for example sponge, can be accomplished either by inserting a compressed sponge through the ureter and then injecting the drug into it, or by inserting a sponge already pre-loaded with the drug. Another method of introducing the sponge into the kidney is suprapubic delivery, usually performed using an endoscope or trocar. The sponge can be inserted into any part of the kidney. In some cases, it can be inserted in the region of the cerebral cortex or adjacent to the papilla, or at one (or at least one) end of the ureter, or to any one or more calyxes of the kidney.

As disclosed above, biodegradable sponges can be made, for example, from collagen, gelatine or any other biodegradable material. The amount of drug can usually be significantly lower than with the systemic use. As mentioned above, intracavitary or intraluminal treatment is superior to systemic treatment for several reasons such as lower dose, no need to expose the entire body to the drug, and lower peak dose, hence fewer expected side effects.

The biocompatible and biodegradable article of the drug delivery system of the present invention is selected from an open-cell sponge, pre-loaded with a drug or without the drug, and the drug can be injected after the compressed sponge expands inside the kidney. It can be a dense sponge, the bubbles of which are filled with the drug. The drug may be a combination of two or more drugs.

The pharmacokinetics of a drug can be controlled by using the biodegradation time of the sponge and monitoring the diffusion of the drug from the sponge. Biodegradation can be controlled by varying the degree of cross-linking of the sponge material. It is known from the literature that the biodegradation time of a collagen or gelatine sponge can range from several hours to many months.

Controlling the release of the drug from the sponge by diffusion can be done by:
1) Mixing the drug with a gel, which will harden the drug and sustain its release. A non-limiting example of such a gel would be a reverse gelation gel. This means that at room temperature it has a low viscosity, similar to water, so it can be easily mixed with medication and easily administered into a sponge after it expands inside the body. It then hardens at body temperature, preventing diffusion of the drug.
2) Coating with a thin layer that is opaquer for the drug to penetrate through it. Such a layer may, for example, be made of very thin perylene, in some embodiments having a thickness of 1 to 10 microns. The sponge can be coated with other polymers, such as polyvinyl alcohol (PVA).
3) The outer surface of the sponge can be made by additional cross-linking of the same material, by creating a sponge with a smaller pore size, or by filling the outer layer of the sponge with a sealing material such as paraffin or fat matrix.
4) The geometry of the sponge also determines diffusion. Such a ball, having a low surface to volume ratio, will have slower diffusion than a cylindrical sponge.

The size of the sponge when enlarged (unfolded) essentially varies. In some embodiments, where a short release time and lower dosage is required, it may be 1 cubic centimetre (cm³). In another embodiment, these can be several cubic centimetres. One of the most attractive sponge shapes is the cylindrical shape, which can be compressed to a diameter of less than 5 millimetres, allowing it to be pushed through a catheter or endoscope. The length of the sponge may vary depending on the amount of medication needed and the duration of biodegradation.

As described above, liquid drugs are used in the drug delivery system of the present invention. However, due to some limitations of the size of the sponge and the need to dissolve the drug in a relatively high amount of water, a drug suspension is used in another embodiment of the invention. Other formulations like microspheres, liposome coatings, and other modes of encapsulation can be used as well.

In a certain embodiment, any biocompatible gel is used in the system and method of the present invention. In a specific embodiment, a gel used in the present invention, is the gel that at 5° C. has a low viscosity, while at body temperature, it is solidified. Different amounts of Co-polymers of f127 HPMC and glycerol can be used to fine-tune the gel properties.

### Preparation of Gel for the System and Method of the Present Invention

The prepared gel for use in the system and method of the present invention possesses reverse rheology properties. Its gel point is approximately 18-22° C.

Materials

| | | | |
|---|---|---|---|
| DW | 763.7 | mL | |
| PEG 500 | 9.0 | g | (0.9%) |
| Glycerol | 0.5 | g | (0.05%) |
| HPMC | 1.8 | g | (0.18%) |
| Pluronic F-127 | 225.0 | g | (22.5%) |
| Ice for an ice bath | 1,000.0 | g | |

Equipment: Basic glass equipment (beaker, Erlenmeyer, pipettes, graduated cylinder), micropipettes, magnetic stirrer with heating, overhead stirrer, Ultra-Turrax homogeniser, one 2-L flask with screw cap, two 500-mL flasks with screw cap, glass container for ice bath, and semi-analytical balances.
Measuring Instruments: Digital thermometer, viscometer and spindles (#1 to #4).

### Gel Preparation Procedure

Pour the DW into a 2-L beaker. Place the beaker in an ice bath, below an overhead stirrer. Activate the stirrer at medium speed (about 250 rpm) and add the PEG 500 and the glycerol. Continue stirring for 5 minutes. Increase stirrer speed to high (about 800 rpm) and very gradually add the HPMC (the whole amount in about 8-10 minutes). If an excess of foam is created, slow the stirrer speed to 100 rpm for 5-10 min without adding HPMC, then increase speed again to 800 rpm and continue adding the rest of the HPMC, very gradually. Allow the stirring to continue for 30 minutes. Check the solution temperature. Allow it to go below 8° C. before commencing next step. Add very gradually the whole amount of Pluronic during appx 1 hour. Allow the stirring to continue for 1 hour. Monitor temperature and don't let it go beyond 8° C.

Place the beaker with its contents and the ice bath under the homogeniser. Submerge its head in the solution and activate the instrument to about half its maximum speed for 10 minutes. If there is excess foaming stop the homogeniser, let the solution “rest” for 5 minutes and continue until the 10 minutes are completed. Bottle the solution into a 2-L flask previously sterilised with boiling water. Leave the gel to “rest” for 3-4 days in the refrigerator at 4° C.

### Gel Testing

### Parameters to be Tested

### Short Term

Appearance (homogeneity, transparency, foam, biological contamination, etc.)
pH
Gel point
Viscosity in the range 4-40° C.
   Longer Term—to be Performed Visually by the Customer after 2 Weeks from Delivery
Stability (no phase separation)
Biological contamination (no growth of microorganisms)

### Results Obtained

Gel appeared as stable, homogeneous, colourless, odourless and transparent. pH value was determined with a pH-meter to obtain pH 8.9 at 8° C. Gel point was determined with the magnetic stirrer method to obtain 19.6° C. Viscosity was measured in a Brookfield-type instrument with spindles interchanged according to viscosity range. Spindle speed was 250 rpm. The table summarises the results.

| T [° C.] | η [mPa × s] |
|---|---|
| 4.0 | 33.3 |
| 10.0 | 65.1 |
| 12.6 | 79.1 |
| 14.1 | 86.6 |
| 16.0 | 360 |
| 17.1 | 451 |
| 18.1 | 653 |
| 18.7 | 1025 |
| 19.2 | 2619 |
| 19.6 | 5780 |
| 20.0 | 9080 |
| 20.8 | 11450 |
| 21.4 | 13060 |
| 25.2 | Very high |
| 37.0 | Very high |
| 40.1 | Very high |

1. A method for treating a condition or disease of a body cavity of a patient in need thereof, the method comprising an intracavitary or intraluminal insertion into the body cavity a drug delivery system configured to release at least one drug into the body cavity; said drug delivery system comprises:
(1) An insertion aid suitable for insertion of a biocompatible and biodegradable article into the body cavity, and
(2) The biocompatible and biodegradable article within said insertion aid;
wherein said article is either preloaded with at least one drug before placement inside the insertion aid, or placed inside the insertion aid without the drug and then filled with the drug after insertion into the body cavity; said article is deformable and selected from the group consisting of:
(a) A deformable article designed to be compressed to a size suitable for placing the article into the insertion aid and through the insertion aid into the body cavity of the patient, wherein the article expands to an expanded retention shape upon release from the insertion aid due to its inherent elasticity; and
(b) A non-deformable article designed to be compressed to a size suitable for placing the article into the insertion aid and through the insertion aid into the body cavity of the patient, wherein the article is preloaded with the drug prior to insertion and expands to an expanded retention shape upon contact with a liquid medium inside the body cavity; and
wherein the expanded retention shape is suited to retain the article within the body cavity of the patient for an extended release of the drug; said extended release of the drug is governed by a degradation rate of the article, dissolution rate or a diffusion rate of the drug into the liquid medium inside the body cavity, or by pH, pressure, or temperature changes.

2. The method of claim 1, wherein:
the condition or disease is urinary incontinence in a patient;
the biocompatible and biodegradable article further comprises a magnetic or ferromagnetic element, or a magnet, and has a molecular weight of about 1, such that it is buoyant in urine; and
the method further comprises placing an absorbent pad with an embedded magnet, or magnetic or ferromagnetic element, near the urethral orifice, wherein:
when the article is within the bladder and the absorbent pad is positioned near the urethra, a magnetic force between the pad and the article attracts the article towards the urethral orifice to seal the urethra and prevent urine leakage; and
when the article is within the bladder and the absorbent pad is moved away from the urethral orifice, the magnetic force is reduced, allowing the article to move away from the urethral orifice and enabling urine to flow.

3. The method of claim 1, wherein the condition is cancer.

4. The method of claim 1, wherein the body cavity is the renal pelvis of a kidney, and the disease is transitional cell carcinoma, or wherein the body cavity is the bladder, and the condition or disease is selected from the group consisting of urinary incontinence, overactive bladder, underactive bladder, interstitial cystitis, urinary tract infections, nocturia, bladder cancer, and neurogenic bladder.

5. The method of claim 1, wherein the drug is selected from the group consisting of an antineoplastic drug, an anticancer drug, a chemotherapeutic agent, an anti-infective agent, an antimicrobial drug, an antiparasitic agent, an antiviral, a genitourinary system drug, an anti-inflammatory drug, an analgesic, a musculoskeletal system acting drug, a drug acting on the blood and blood-forming organs, an antihemorrhagic, an antithrombotic agent, an antianemia drug, a dermatologic drug, an antifungal, an antiseptic, a gastrointestinal system drug, an anti-obesity drug, an acid-related disorder drug, a metabolism drug, a neurological drug, a respiratory drug including nasal drugs, a cardiovascular drug, an ontological drug, a corticosteroids drug, an analgesic drug, an antiparasitic drug, an anaesthetic drug, botulinum toxin, amoxicillin, ceftriaxone, cephalexin, ciprofloxacin, fosfomycin, levofloxacin, amikacin, piperacillin, nitrofurantoin, trimethoprim, sulfamethoxazole, gentamicin, imipenem, meropenem, progesterone, ceftolozane, cefiderocol, plazomicin, neomycin, kanamycin, paromomycin, bacitracin, vancomycin, colistin, polymyxin, amphotericin, lidocaine, paclitaxel, mitomycin C, gemcitabine, quinolone, fluoroquinolone, nadofaragene firadenovec, enfortumab vedotin, sacituzumab govitecan, brilacidin.

6. The method of claim 1, wherein the drug is selected from the group consisting of a PD-1 inhibitor, an anti-PD-1 antibody, a PD-L2 inhibitor, an anti-PD-L2 antibody, a tyrosine kinase inhibitor, a humanized monoclonal antibody (mAb) h11B6 that exclusively targets hK2, an anti-CTLA-4 monoclonal antibody, and combinations thereof.

7. The method of claim 6, wherein the drug is selected from the group consisting of dostarlimab, pembrolizumab, cetrelimab, nivolumab, ipilimumab and tremelimumab.

8. The method of claim 6, wherein the anti-CTLA-4 monoclonal antibody is administered intravesically and in combination with an anti-PD-1 antibody.

9. The method of claim 1, wherein said article is coated with a biocompatible and biodegradable or non-biodegradable coating comprising at least one opening for a zero-order drug release, and the drug is released from the article by diffusion into the body cavity, optionally at a constant rate, over an extended-release duration of at least one day and up to 30 days, with article degradation commencing after the extended-release.

10. The method of claim 9, wherein said coating is a thin layer of a biocompatible and non-biodegradable coating material suitable for sloughing, for example, PTFE, silicone, polyurethane, polycarbonate and polysiloxane, with a thickness from about 5 μm to about 50 μm, and weight from about 1 mg to about 5 mg.

11. The method of claim 9, wherein said coating is a layer of a biocompatible and biodegradable coating material, for example, parylene, poly(lactic acid) (PLA), poly(lactic-co-glycolic acid) (PLGA), polycaprolactone (PCL), poly(ε-caprolactone)-based copolymers, chitosan, gelatine, polyhydroxyalkanoates (PHA), polyethylene glycol (PEG), hyaluronic acid, polyglactin, collagen, alginate and carrageenan.

12. The method of claim 1, wherein the article is a compressible and absorbent sponge, optionally comprising a plurality of biodegradable nanosponges, where the nanosponges are nanosized drug carriers with a three-dimensional structure formed by crosslinking polymers, such as beta-cyclodextrins, alginates, carboxymethyl cellulose, chitosan, carrageenans, cross-linked cellulose nanofibers, collagen, gelatin, or combinations thereof, said nanosponges are adapted to provide a controlled drug release pattern with targeted drug delivery; and
wherein said article is adapted to:
absorb a drug;
be compressed and inserted into an insertion aid before the insertion aid is placed in the body cavity;
be released from the insertion aid into the body cavity upon insertion of the insertion aid;
expand within the body cavity upon contact with a liquid medium or through its inherent elasticity; and
release the drug into the liquid medium within the body cavity.

13. The method of claim 1, wherein the drug is mixed with a gel, for example, a reverse gelation biodegradable gel, for controlling viscosity.

14. The method of claim 1, wherein the article is comprised of chemical compounds selected from the group consisting of beta-cyclodextrins, alginates, carboxymethyl cellulose, chitosan, carrageenans, cross-linked cellulose nanofibers, and collagen, or combinations thereof.

15. The method of claim 1, wherein the article is a compressible sponge, and the method further comprises compressing the sponge and pushing the sponge, while it is compressed, into the insertion aid before inserting the insertion aid into the body cavity.

16. The method of claim 1, wherein the article comprises a core material, for example, foam, porous solid, liquid, and gaseous material, having a specific gravity that is less than a specific gravity of water, the material allowing for the article to be buoyant in aqueous fluids.

17. A drug delivery system for an intracavitary or intraluminal delivery of a drug into a body cavity of a patient comprising:
(1) an insertion aid for inserting a biocompatible and biodegradable article into the body cavity; and
(2) the article within said insertion aid;
wherein the article is deformable and selected from:
(a) a compressible article expandable to a retention shape upon release from the insertion aid; or
(b) a non-deformable article preloaded with drug and expandable to a retention shape upon contact with a liquid medium within the body cavity; and
wherein:
(i) the article is either preloaded with at least one drug, or loaded with the drug after insertion; and
(ii) the retention shape retains the article for extended drug release governed by the article's degradation rate, the drug's dissolution or diffusion rate, or by changes in pH, pressure, or temperature.

18. The drug delivery system of claim 17, wherein the body cavity is the renal pelvis of a kidney, and the disease is transitional cell carcinoma, or wherein the body cavity is the bladder, and the condition or disease is selected from the group consisting of interstitial cystitis, urinary tract infections, nocturia, bladder cancer, and neurogenic bladder.

19. The drug delivery system of claim 17, wherein the drug is selected from the group consisting of an antineoplastic drug, an anticancer drug, a chemotherapeutic agent, an anti-infective agent, an antimicrobial drug, an antiparasitic agent, an antiviral, a genitourinary system drug, an anti-inflammatory drug, an analgesic, a musculoskeletal system acting drug, a drug acting on the blood and blood-forming organs, an antihemorrhagic, an antithrombotic agent, an antianemia drug, a dermatologic drug, an antifungal, an antiseptic, a gastrointestinal system drug, an anti-obesity drug, an acid-related disorder drug, a metabolism drug, a neurological drug, a respiratory drug including nasal drugs, a cardiovascular drug, an ontological drug, a corticosteroids drug, an analgesic drug, an antiparasitic drug, an anaesthetic drug, botulinum toxin, amoxicillin, ceftriaxone, cephalexin, ciprofloxacin, fosfomycin, levofloxacin, amikacin, piperacillin, nitrofurantoin, trimethoprim, sulfamethoxazole, gentamicin, imipenem, meropenem, progesterone, ceftolozane, cefiderocol, plazomicin, neomycin, kanamycin, paromomycin, bacitracin, vancomycin, colistin, polymyxin, amphotericin, lidocaine, paclitaxel, mitomycin C, gemcitabine, quinolone, fluoroquinolone, nadofaragene firadenovec, enfortumab vedotin, sacituzumab govitecan, brilacidin, PD-1 inhibitor, anti-PD-1 antibody, PD-L2 inhibitor, anti-PD-L2 antibody, tyrosine kinase inhibitor, humanized monoclonal antibody (mAb) h11B6 that exclusively targets hK2, an anti-CTLA-4 monoclonal antibody, dostarlimab, pembrolizumab, cetrelimab, nivolumab, ipilimumab, tremelimumab. and combinations thereof.

20. A kit for intracavitary or intraluminal drug delivery and sustained release, comprising the drug delivery system of claim 1, and instructions for manual or electronic use.
